(19) United States
(12) Patent Application Publication
(10) Pub. No.: US 2026/0232851 A1
(43) Pub. Date: Aug. 13, 2026

(54) METHODS FOR THE DETECTION AND DIAGNOSIS OF CANCERS ASSOCIATED WITH OVEREXPRESSION OF PSMA RECEPTOR

(71) Applicant: CLARITY PHARMACEUTICALS LIMITED, Eveleigh NSW (AU)

(72) Inventors: Eva LENGYELOVA, Eveleigh NSW (AU); Robert MILLER, Eveleigh NSW (AU); Louise EMMETT, Eveleigh NSW (AU)

(21) Appl. No.: 19/156,214

(22) PCT Filed: Feb. 14, 2024
(86) PCT No.: PCT/AU2024/050097

§ 371 Date: Aug. 13, 2025

(30) Foreign Application Priority Data
Feb. 14, 2023 (AU) 2023900356

(51) Int. Cl. A61K 51/08; A61K 51/04; A61P 35/00

(52) U.S. Cl. CPC A61K 51/088; A61K 51/0482; A61P 35/00

(57) ABSTRACT

Abstract Of The Disclosure.

### FIELD

The present invention relates to methods for the detection and diagnosis of a cancer related to overexpression of prostate-specific membrane antigen.

### BACKGROUND

Prostate cancer is the second most frequent malignancy in men worldwide and is the second most common cancer, accounting for 9.5% of all new cancers in 2018. The occurrence of prostate cancer varies and correlates with age, with incidence between 30% of males aged 40 to 50 years old and 50% to 80% of males aged 80 and over. At initial presentation, 80% of patients have local disease, 12% have regional disease and 4% have metastatic disease. Although the 5-year survival rate for patients with local or regional prostate cancer is 99%, the survival rate drops to about 30% in the case of metastatic disease.

Prostate-specific membrane antigen (PSMA) is a type II transmembrane glycoprotein that is expressed in normal, benign and malignant prostate tissues. The expression of the PSMA membrane protein increases with aggressiveness of the prostate tumour, the presence of metastatic disease and recurrence of the cancer. For example, expression of the PSMA membrane protein is 100- to 1000-fold higher in prostatic adenocarcinoma than in benign prostate tissue and increases with androgen deprivation, with levels of the protein highest in high-grade and castration resistant prostate cancer. While the level of PSMA expression is correlated with development of disease, there are cases of prostate cancer where increased expression of PSMA is not observed in biopsies.

Current approaches for the diagnosis of prostate cancer includes the combination of analysis of prostate-specific antigen (PSA) in blood, imaging using positron emission tomography (PET) and biopsy of prostate tissue. Imaging via PET requires the use of an appropriate radiotracer. ⁶⁸Ga-PSMA-11 is one of the most widely used radiotracers for PET imaging of prostate-specific membrane antigen (PSMA) positive lesions in men with prostate cancer and is part of standard clinical practice at many centres. While PET imaging can be achieved using current radiotracers, the images obtained have limited resolution. This means that small lesions may be missed and lesions that are close to organs that are involved in excretion of the radiotracer (e.g. bladder) may not be sufficiently delineated and identified as being cancerous. Where such lesions are missed, the subsequent delay in treatment may lead to poorer patient outcomes.

There remains a need for methods of diagnosis for prostate cancer that have greater sensitivity and allow for better delineation of cancerous tissue from healthy tissue in patients.

### SUMMARY OF THE INVENTION

According to an aspect, the present invention provides a method for the detection and/or diagnosis of a cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu;

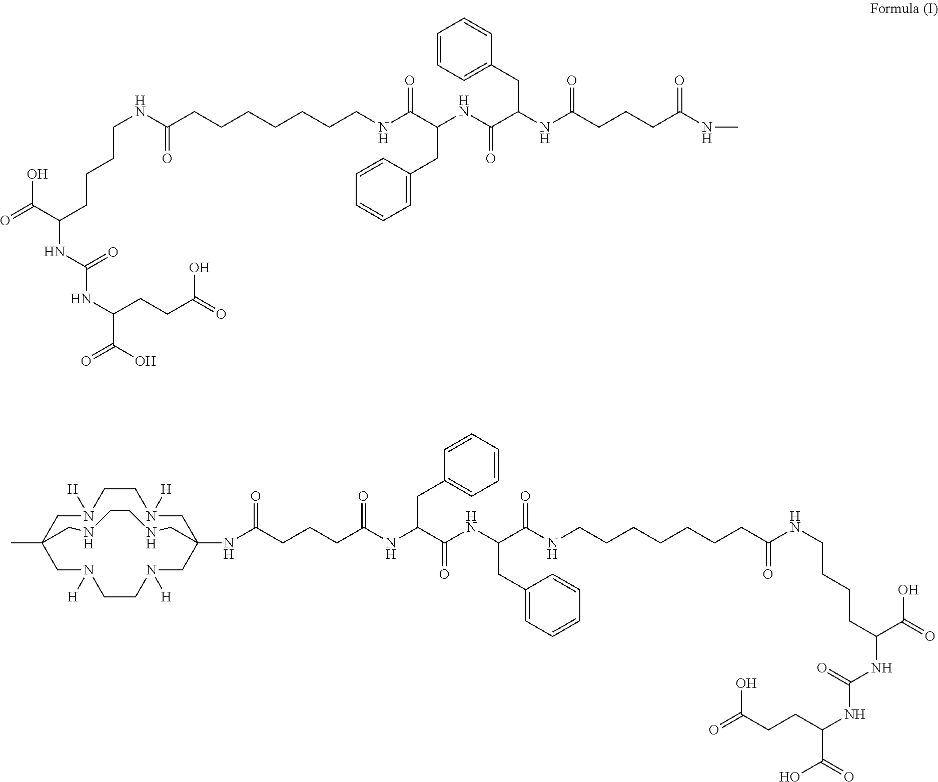

ii) imaging of the subject by PET imaging at a time of at least about 8 hours after administration of the compound of Formula (I).

According to an aspect, the present invention provides a method for the detection and/or diagnosis of a cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu; and

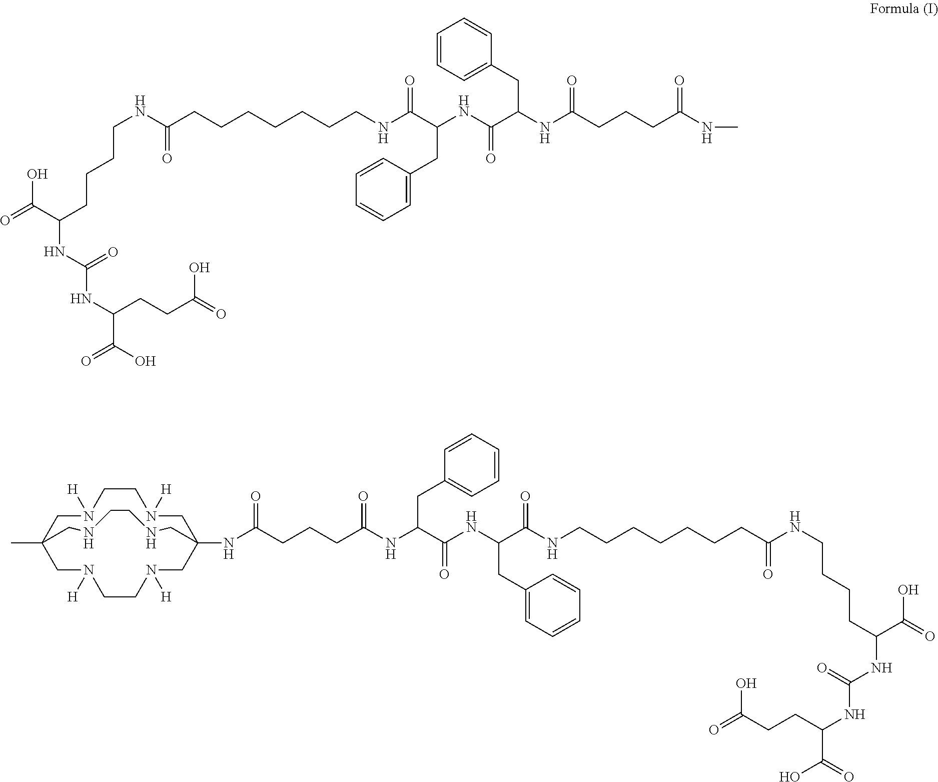

ii) imaging of the subject by PET imaging; and
iii) determining the tumor-to-background ratio (TTBR) of one or more lesions detected by imaging in step ii);
   wherein the tumor-to-background ratio (TTBR) of a lesion associated with the cancer in the subject is more than about 40.

According to an aspect, the present invention provides a method for the detection and/or diagnosis of a cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu;

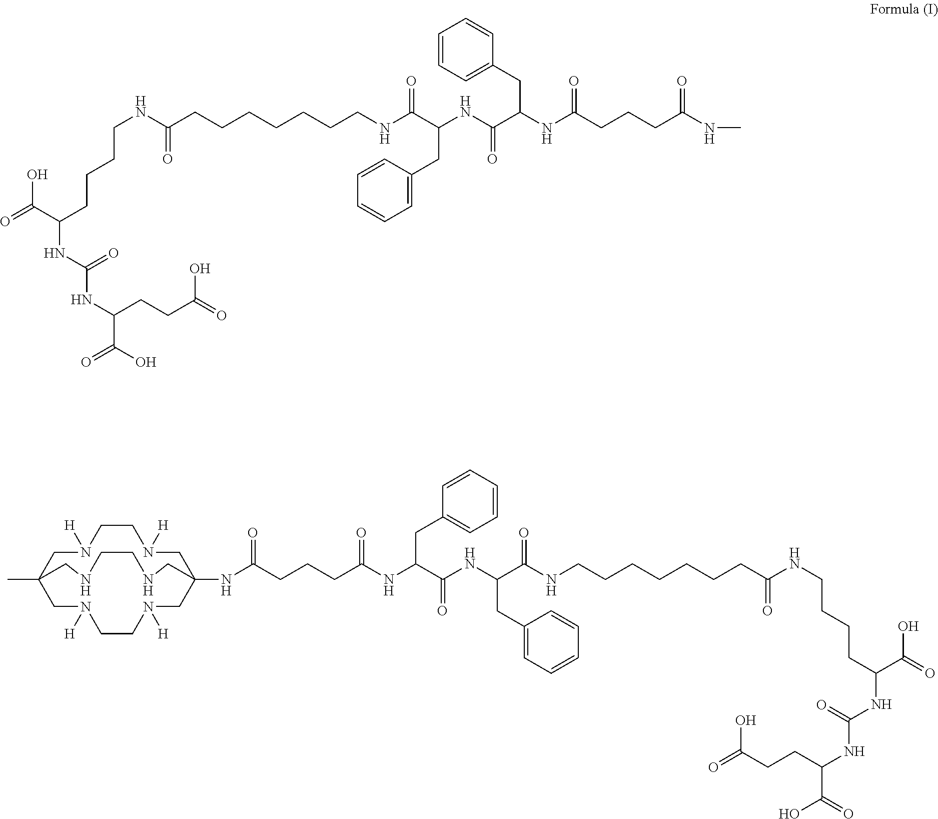

ii) imaging of the subject by PET imaging at a time of at least about 8 hours after administration of the compound of Formula (I);
iii) determining the tumor-to-background ratio (TTBR) of a lesion visualised by imaging in step ii);
   wherein the tumor-to-background ratio (TTBR) of a lesion associated with the cancer in the subject is more than about 40.

According to another aspect, the present invention provides a method for imaging a lesion in a cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu;

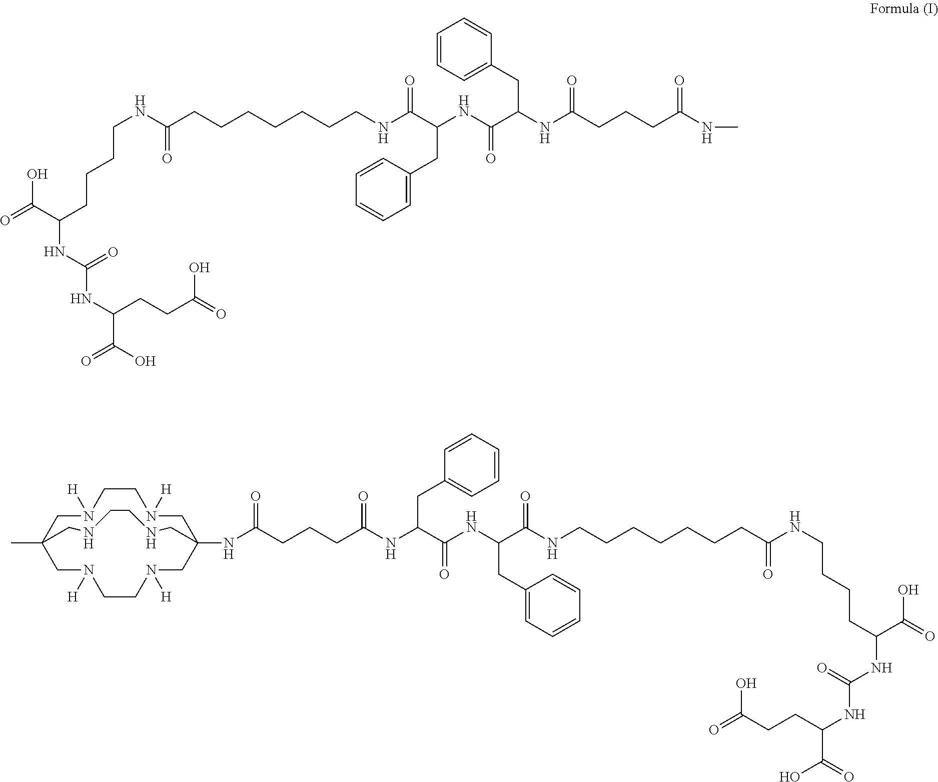

ii) imaging of the subject by PET imaging;
iii) determining the tumor-to-background ratio (TTBR) of one or more lesions detected by imaging in step ii);
   wherein the lesion is characterised by a tumor-to-background ratio (TTBR) of more than about 40.

According to another aspect, the present invention provides a method for imaging a lesion in a cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu;

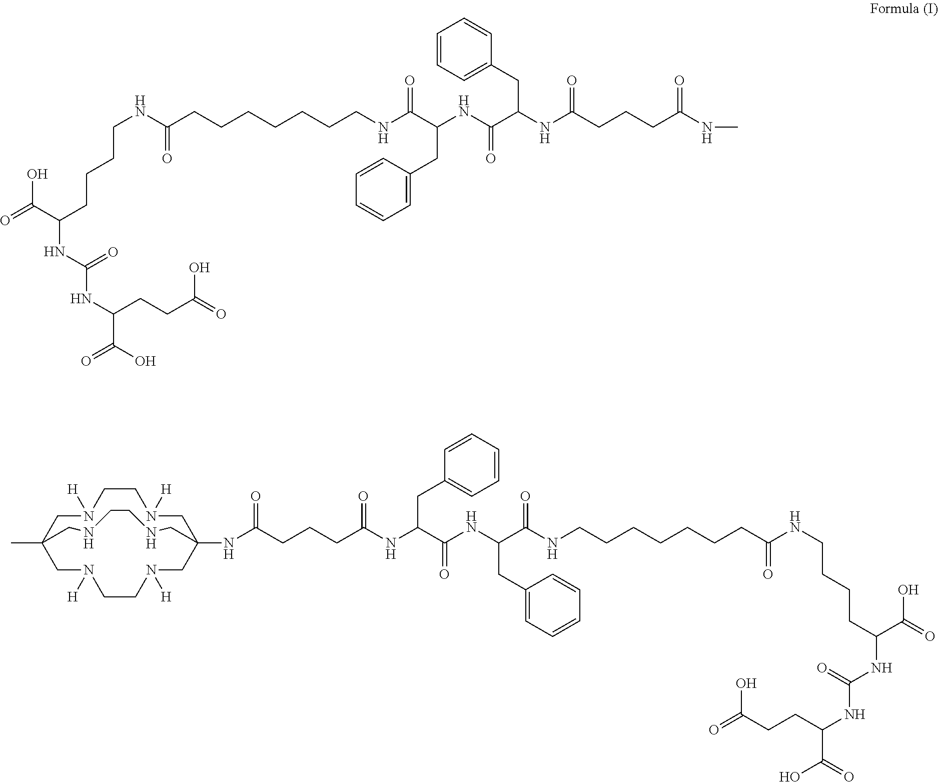

ii) imaging of the subject by PET imaging at a time of at least about 8 hours after administration of the compound of Formula (I);
iii) determining the tumor-to-background ratio (TTBR) of one or more lesions detected by imaging in step ii);
   wherein the lesion is characterised by a tumor-to-background ratio (TTBR) of more than about 40.

According to an aspect, the present invention provides a method for the detection and/or diagnosis of a cancer associated with overexpression of PSMA in a subject in need thereof, the method comprising:
i) administering to the subject a dose of between about 100 to about 300 MBq of a compound of Formula (I) or a salt thereof complexed with ⁶⁴Cu; and
ii) imaging of the subject by PET imaging at least once between about 30 minutes and about 36 hours after administration of the compound of Formula (I);

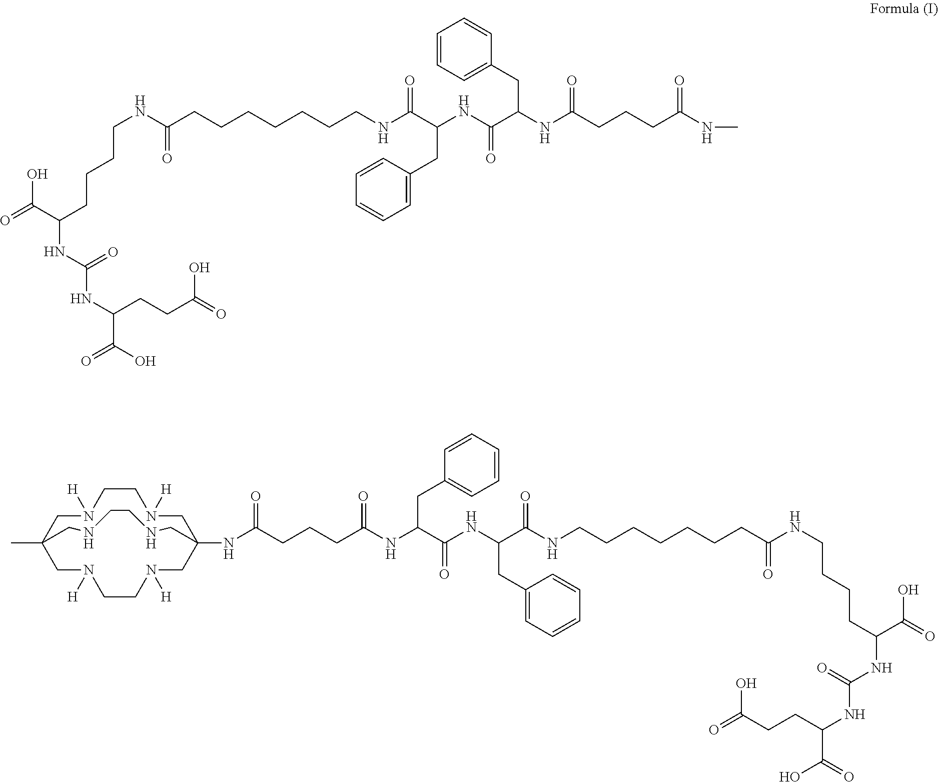

wherein the image of the cancer is at a higher resolution relative to ⁶⁸Ga-PSMA-11 administered under standard of care conditions.

In certain embodiments, the dose of the compound of Formula (I) or a salt thereof complexed with ⁶⁴Cu is about 100 MBq. In other embodiments, the dose of the compound of Formula (I) or a salt thereof complexed with ⁶⁴Cu is about 150 MBq. In other embodiments, the dose of the compound of Formula (I) or a salt thereof complexed with ⁶⁴Cu is about 200 MBq. In other embodiments, the dose of the compound of Formula (I) or a salt thereof complexed with 64 Cu is about 300 MBq.

In certain embodiments, imaging of the subject occurs about 8 hours after administration of the compound of Formula (I) complexed with ⁶⁴Cu. In certain embodiments, imaging of the subject occurs about 10 hours after administration of the compound of Formula (I) complexed with ⁶⁴Cu. In certain embodiments, imaging of the subject occurs about 12 hours after administration of the compound of Formula (I) complexed with ⁶⁴Cu. In certain embodiments, imaging of the subject occurs about 16 hours after administration of the compound of Formula (I) complexed with ⁶⁴Cu. In certain embodiments, imaging of the subject occurs about 18 hours after administration of the compound of Formula (I) complexed with ⁶⁴Cu. In certain embodiments, imaging of the subject occurs about 20 hours after administration of the compound of Formula (I) complexed with ⁶⁴Cu. In certain embodiments, imaging of the subject occurs about 24 hours after administration of the compound of Formula (I) complexed with ⁶⁴Cu. In certain embodiments, imaging of the subject occurs about 28 hours after administration of the compound of Formula (I) complexed with ⁶⁴Cu. In certain embodiments, imaging of the subject occurs about 30 hours after administration of the compound of Formula (I) complexed with ⁶⁴Cu. In certain embodiments, imaging of the subject occurs about 36 hours after administration of the compound of Formula (I) complexed with ⁶⁴Cu.

In certain embodiments, the tumor-to-background ratio (TTBR) is more than about 45. In some embodiments, the TTBR is more than about 50. In some embodiments, the TTBR is more than about 55. In some embodiments, the TTBR is more than about 60. In some embodiments, the TTBR is more than about 65. In some embodiments, the TTBR is more than about 70. In some embodiments, the TTBR is more than about 75. In some embodiments, the TTBR is more than about 80. In some embodiments, the TTBR is more than about 85. In some embodiments, the TTBR is more than about 90. In some embodiments, the TTBR is more than about 95. In some embodiments, the TTBR is more than about 100. In some embodiments, the TTBR is more than about 105. In some embodiments, the TTBR is more than about 110. In some embodiments, the TTBR is more than about 115. In some embodiments, the TTBR is more than about 120. In some embodiments, the TTBR is more than about 125. In some embodiments, the TTBR is more than about 130. In some embodiments, the TTBR is more than about 140. In some embodiments, the TTBR is more than about 145. In some embodiments, the TTBR is more than about 150. In some embodiments, the TTBR is in the range of between about 40 to about 150. In other embodiments, the present invention also contemplates a TTBR in a range between the aforementioned values.

According to another aspect, the present invention provides a method for the detection and/or diagnosis of a cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu;

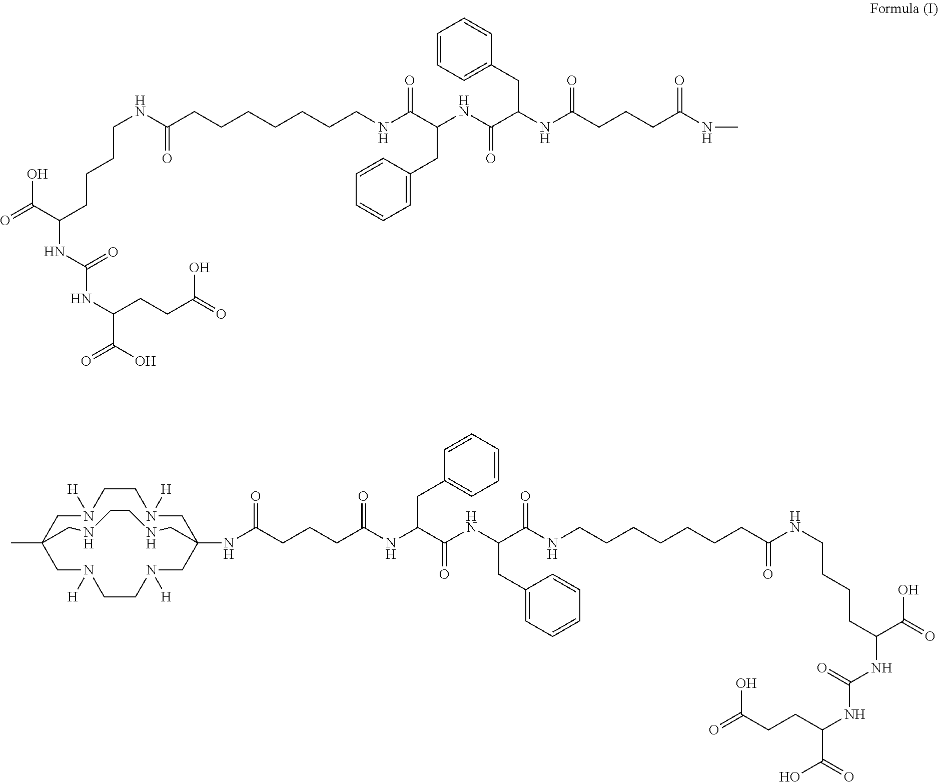

ii) imaging of the subject by PET imaging at a time of at least about 8 hours after administration of the compound of Formula (I);
   wherein the image of the cancer is at a higher resolution relative to ⁶⁸Ga-PSMA-11 administered under standard of care conditions.

According to another aspect, the present invention provides a method for the detection and/or diagnosis of a cancer associated with overexpression of PSMA in a subject in need thereof, the method comprising:
i) administering to the subject a dose of between about 100 to about 300 MBq of a compound of Formula (I) or a salt thereof complexed with ⁶⁴Cu; and
ii) imaging of the subject by PET imaging between about 2 hours and about 4 hours after administration of the compound of Formula (I);

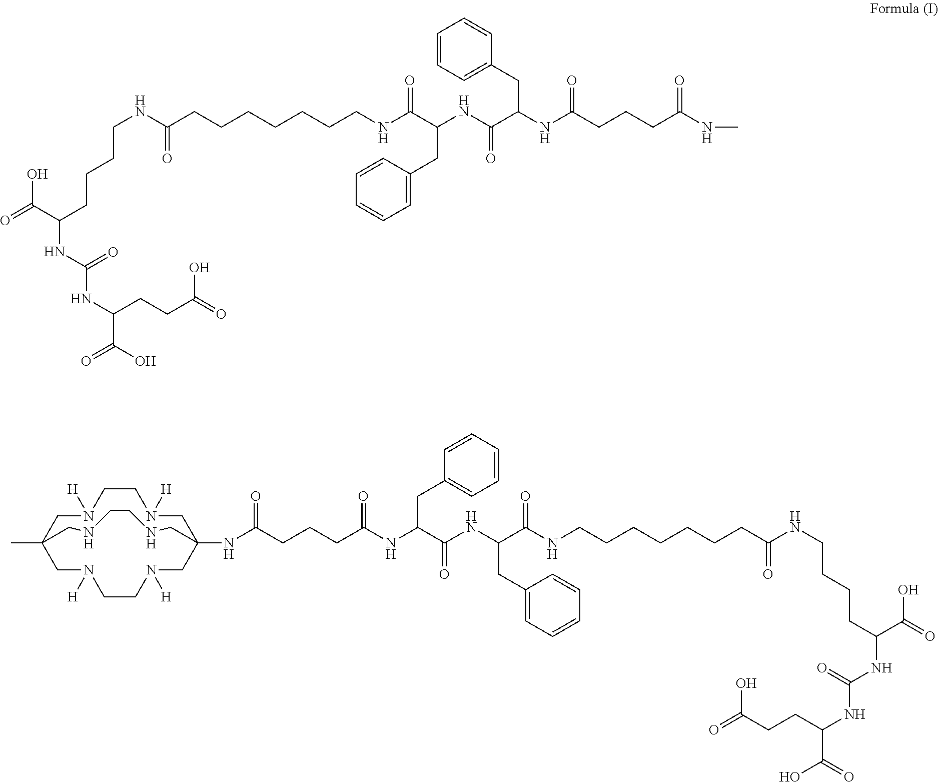

wherein the image of the cancer is at a higher resolution relative to ⁶⁸Ga-PSMA-11 administered under standard of care conditions.

The present inventors have found that the images obtained according to a method of the present invention allows for images of greater resolution to be obtained when compared to methods employing a standard-of-care (SOC) radiotracer for the imaging of cancers overexpressing PSMA.

The SOC radiotracer for PET imaging of cancers that overexpress PSMA is a ⁶⁸Ga-containing compound. In certain embodiments, the ⁶⁸Ga-containing compound is ⁶⁸Ga-PSMA-11, as depicted below:

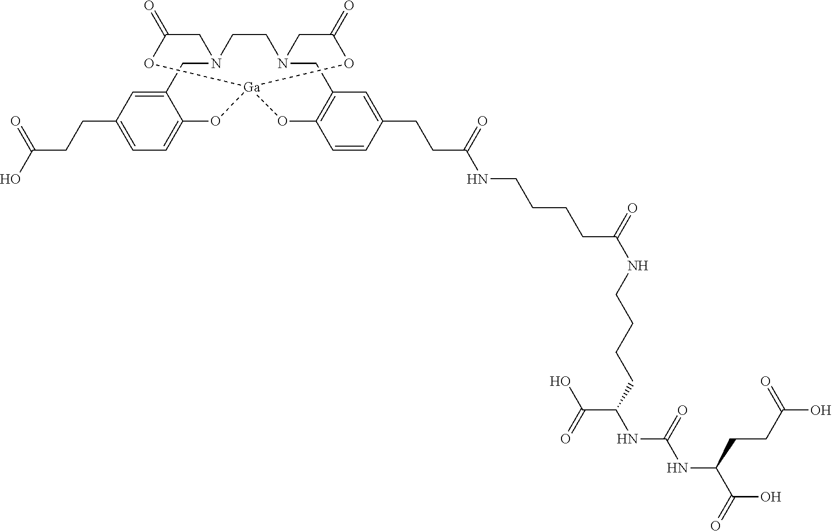

Another radiotracer used for PET imaging of cancers that overexpress PSMA is a ¹⁸F containing compound. In certain embodiments, the 18-F-containing compound is ¹⁸F-DCFPyL (Pylarify®), as depicted below:

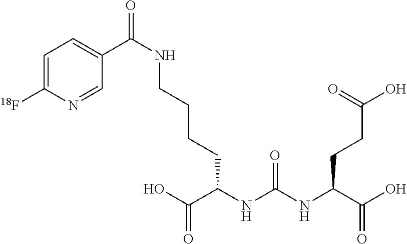 [SMILES: O=C(O)CC[C@H](NC(=O)N[C@@H](CCCCNC(=O)c1ccc([18F])nc1)C(=O)O)C(=O)O]

Images obtained via PET show the uptake and localisation of the radiotracer at sites in the body. The radiotracer comprising a compound capable of binding a target (e.g. a PSMA receptor) and a positron-emitting radioisotope localises at sites that overexpress the PSMA receptor and are indicative of a cancer. Since the radioisotope decays with the emission of a positron, detection of the positrons emitted correspond to the location of the cancer. PET images can then be overlaid with other imaging modalities that reveal structural information, for example, CT scans, to provide information regarding the location of the cancer. The intensity of the signals at a particular region of interest indicate to a reader the relative amount of the radiotracer that has accumulated at a particular site, with signals of greater intensity (or “brightness”) owing to more accumulation of the radiotracer and correlate with the presence of a cancer. One measure of intensity is the standardized uptake value (SUV), which is defined as the ratio of radioactivity in a region of interest to the injected radioactivity across the whole body of the subject. Higher SUVs indicate areas of greater “brightness” and hence radioactivity. Greater contrast between regions of interest, i.e. higher SUVs, leads to images of higher resolution being obtained. SUVs across images may be compared by first determining particular values including maximum uptake values (SUVmax), mean uptake values (SUVmean), median uptake values (SUVmedian), values relative to body weight of the patient (SUVbw), values relative to BMI of the patient (SUVBMI) and the like. Another measure of signal intensity is the tumor-to-background ratio (TTBR), which is the ratio of a signal attributed to the tumor associated with the cancer to a signal attributed to the “background” or healthy tissue of the same subject. The use of a TTBR as a manner of comparing the uptake of the compound of Formula (I) may show significant advantages over other measures of intensity, e.g. SUVmax and SUVmean, as the calculation of the TTBR takes into account the background intensity of the image.

In order to correctly identify a cancer on the basis of images obtained by PET imaging, images of a sufficient resolution and/or contrast are required. The present inventors have found that the methods comprising the administration of a compound of Formula (I) complexed with ⁶⁴Cu provide images of a higher resolution and/or contrast when compared to a SOC radiotracer, for example, a radiotracer comprising a ⁶⁸Ga or ¹⁸F radioisotope. Images of greater contrast may be obtained with the administration of the compound of Formula (I) complexed with ⁶⁴Cu, since the present inventors have now shown that localisation of the compound of Formula (I) at tumor sites overexpressing the PSMA receptor and subsequent imaging occurs at a higher rate than other radiotracers, i.e. the ratio of the compound at the tumor site and the background is higher. Since images of a higher resolution and/or contrast, specifically contrast, may be obtained, cancerous lesions that would be otherwise overlooked (e.g. due to little localisation of the radioisotope complex and subsequent low TTBR values obtained) may be detected. Higher resolution images also allow smaller cancerous lesions to be detected, particularly smaller lesions that are in close proximity to each other or to organs that are in involved in clearance of the product (e.g. bladder and kidneys).

Without wishing to be bound by theory, the present inventors believe that the higher resolution and/or contrast of images obtained when using the compound of Formula (I) complexed with ⁶⁴Cu is due in part to the longer half-life and shorter positron range (t_{1/2}=12.7 hours; mean positron range=0.56 mm for ⁶⁴Cu) when compared to a SOC radiotracer ⁶⁸Ga-PSMA-11 (t_{1/2}=68 minutes; mean positron range=3.5 mm for ⁶⁸Ga) or ¹⁸F-DCFPyL (t_{1/2}=109 minutes, mean positron range <1 mm for ¹⁸F). The 12.7 hour half-life of ⁶⁴Cu allows the central manufacturing of the radiolabelled compound of Formula (I) with a product shelf-life of up to 2 days. The longer half-life enables PET imaging from 1 to 72 hours after administration which offers greater flexibility in terms of scheduling of patients and may translate into detection of additional lesions due to increased Standardized Uptake Values (SUV) in the lesions relative to background following biological clearance of the tracer from organs over time. Higher resolution of the images obtained according to the methods disclosed herein is also due to the nature of the compound of Formula (I), i.e. the compound of Formula (I) contains two moieties that are able to target and bind the receptor of interest. Furthermore, the physical properties of the compound of Formula (I) allows for increased binding at the target site and sufficient clearance of the radiotracer in order to provide increased contrast between tumour sites and healthy tissue.

Positron range refers to the distance travelled by an emitted positron before losing all its kinetic energy and being annihilated with an electron. As a result, the annihilation points form a uniform distribution around the actual emission point, thereby causing image blurring for positron ranges larger than the system's intrinsic spatial resolution. Since the ⁶⁸Ga isotope has a much larger positron range, the distribution of positron-electron annihilation events is subsequently larger and the area over which these events are detected (and imaged) is also larger and more diffuse. This leads to images of a lower resolution.

A common measure of intensity in PET imaging is the maximum standardized uptake value (SUVmax). In certain embodiments, the maximum SUV (SUVmax) obtained from images produced according to the methods disclosed herein is higher than the value obtained from comparable images using a SOC radiotracer. In certain embodiments, the maximum SUV (SUVmax) obtained from images produced according to the methods disclosed herein is higher than the value obtained from comparable images using a SOC radiotracer comprising ⁶⁸Ga. In certain embodiments, the maximum SUV (SUVmax) obtained from images produced according to the methods disclosed herein is higher than the value obtained from comparable images using a SOC radiotracer comprising ¹⁸F. In certain embodiments, the SUVmax attributed to the compound of Formula (I) complexed with ⁶⁴Cu is about 10%, about 15%, about 20%, about 25%, about 30%, about 35%, about 40%, about 45% or about 50% higher than the same value in accordance with a radiotracer comprising ⁶⁸Ga. In certain embodiments, the SUVmax attributed to the compound of Formula (I) complexed with ⁶⁴Cu is about 10%, about 15%, about 20%, about 25%, about 30%, about 35%, about 40%, about 45% or about 50% higher than the same value in accordance with a radiotracer comprising ¹⁸F. As seen in FIG. 4, the SUVmax of lesions imaged in accordance with the methods as disclosed herein (i.e. after administration of ⁶⁴Cu-Sar-bisPSMA) is greater than when the same subject is administered a radiotracer comprising ⁶⁸Ga. The higher SUVmax in images taken in accordance with methods disclosed herein was reported by two different image readers. Given the higher intensity of the signal (SUVmax), the present inventors believe that the methods disclosed herein allow for a more efficient and effective diagnosis of cancers that are associated with the PSMA receptor.

FIG. 7 also shows an increase in the SUVmax of images taken from the same subject, where imaging on Day 0 gave an SUVmax of 20.8 and the same imaging repeated the next day (i.e. Day 1) gave an SUVmax of 50.4. The present inventors believe that the flexibility in imaging a patient after administration of the compound of Formula (I) complexed with ⁶⁴Cu allows for more effective diagnosis of cancers associated with a PSMA receptor, since lesions associated with such cancers that are not visualised by same-day imaging are often missed when currently available ligands and radioisotopes are used. Since imaging of the subject may be performed the following day after administration of the compound of Formula (I), the patient may be more comfortable, which in turn increases patient compliance with the procedure. The present inventors have found that the maximum uptake value is greater when a subject is imaged a day after the compound of Formula (I) complexed with ⁶⁴Cu is imaged. This can be seen in FIG. 8, where imaging of the subject on the day of administration (FIG. 8A) does not clearly visualise a lesion, however repeat imaging the following day (FIG. 8B) successfully identifies a lesion in the same subject.

Another measure of intensity in PET imaging is the mean standardised uptake value (SUVmean). In other embodiments, the mean SUV (SUVmean) obtained from images produced according to the methods disclosed herein is higher than the value obtained from comparable images using a SOC radiotracer comprising ⁶⁸Ga or ¹⁸F. In certain embodiments, the SUVmean attributed to the compound of Formula (I) complexed with ⁶⁴Cu is about 10%, about 15%, about 20%, about 25%, about 30%, about 35%, about 40%, about 45% or about 50% higher than the same value in accordance with a radiotracer comprising ⁶⁸Ga. This can be seen in FIGS. 2 (B) and (D), which clearly shows lesions visualised with a higher intensity, as compared with the same lesions imaged with an existing ⁶⁸Ga protocol in FIGS. 2 (A) and (C). As seen in FIG. 5, the SUVmean of lesions imaged in accordance with the methods as disclosed herein (i.e. after administration of ⁶⁴Cu-Sar-bisPSMA) is greater than when the same subject is administered a radiotracer comprising ⁶⁸Ga. The higher SUVmean in images taken in accordance with methods disclosed herein was reported by two different image readers. Since the mean uptake value (SUVmean) attributed to the methods disclosed herein is higher than in accordance with existing methods, the present inventors believe that the methods herein can provide an improved process for the diagnosis of a cancer associated with PSMA receptors.

The present inventors believe that the PET images obtained according to methods of the present invention have a higher resolution due to the nature of the radioisotope (i.e. ⁶⁴Cu) that is complexed with the compound of Formula (I). As disclosed herein, the methods include imaging of the subject at least once between about 30 minutes and about 36 hours after administration of the radiotracer. In comparison, methods of imaging in accordance with the current SOC model includes the administration of a dose of ⁶⁸Ga-PSMA-11 with radioactivity between 111 MBq and 259 MBq (in accordance with the FDA), with images obtained after a time of about 1 hour. Since the ⁶⁸Ga isotope has a shorter half-life (t_{1/2}), the time between administration and imaging must be optimised to ensure that images of a sufficient quality may be obtained. The present inventors have found that since the ⁶⁴Cu radioisotope used in the methods disclosed herein have a longer half-life, a longer time between administration and imaging is possible. Without wishing to be bound by theory, the present inventors believe that increasing the time between administration of the compound of the radiotracer and subsequent imaging provides greater opportunity for the radiolabelled complex to localise at cancer sites and for any unbound radiotracer to be cleared from the subject. This in turn allows for images of a greater resolution to be obtained, since contrast between the sites of localisation of the radiotracer and background is greater. This can be seen in FIG. 6, which shows the mean ratio of the tumor to background, where the mean ratio is higher in images obtained after administration of ⁶⁴Cu-Sar-bisPSMA when compared to administration of ⁶⁸Ga-PSMA-11.

The present inventors also believe that the higher maximum, mean and median SUV obtained as a result of imaging after administration of the compound of Formula (I) complexed with ⁶⁴Cu is also at least partially attributed to the dimeric nature of the compound of Formula (I). As seen in the structure of Formula (I), the compound contains two lysine-urea-glutamate motifs attached to the sarcophagine chelator via linkers groups. Since the corresponding monomeric compound, i.e. a sarcophagine bearing a single lysine-urea-glutamate motif (and depicted below), shows less uptake and retention when compared to the dimeric compound of Formula (I) disclosed herein.

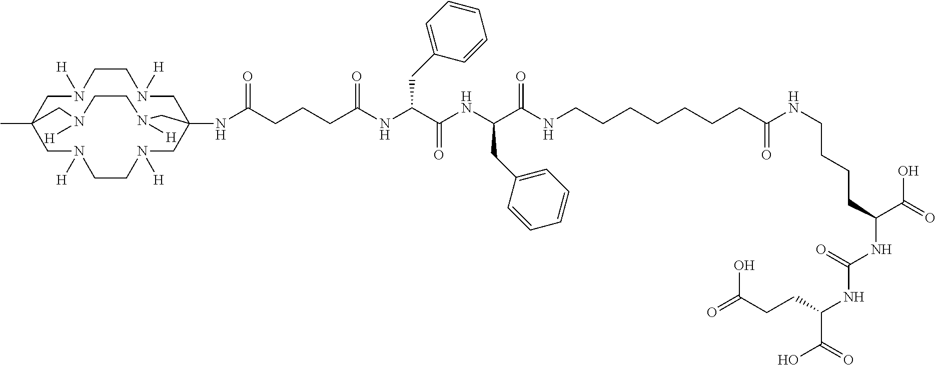 [SMILES: CC12CNCCNCC(NC(=O)CCCC(=O)N[C@H](Cc3ccccc3)C(=O)N[C@H](Cc3ccccc3)C(=O)NCCCCCCCC(=O)NCCCC[C@H](NC(=O)N[C@@H](CCC(=O)O)C(=O)O)C(=O)O)(CNCCNC1)CNCCNC2]

FIG. 1 shows that the compound of Formula (I) has higher uptake and retention at tumors when compared to the corresponding monomeric compound containing a single lysine-urea-glutamate motif.

In some embodiments, the dose of a compound of Formula (I) or a salt thereof complexed with ⁶⁴Cu is about 200 MBq.

In certain embodiments, the cancer is a prostate cancer.

In other embodiments, the cancer is a primary prostate cancer. In some embodiments, the cancer is a prostate cancer associated with biochemical recurrence.

In certain embodiments, the imaging is combined PET/CT imaging.

In some embodiments, imaging of the subject occurs at about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours or about 36 hours after administration of the compound of Formula (I) complexed with ⁶⁴Cu. In other embodiments, imaging of the subject occurs at about 12 hours, about 24 hours or about 36 hours after administration of the compound of Formula (I) complexed with ⁶⁴Cu.

In certain embodiments, the method comprises a further imaging step of the subject by PET imaging. In some embodiments, the further imaging step occurs about 4 hours, about 6 hours, about 8 hours, about 10 hours, about 12 hours, about 16 hours, about 18 hours, about 24 hours, about 30 hours, about 36 hours, about 42 hours, about 48 hours, about 54 hours and/or about 60 hours after the first imaging step.

In certain embodiments, the method further includes the step of MRI imaging or ultrasound imaging.

In certain embodiments, the method further comprises the step of determining the PSA level of the subject.

In certain embodiments, the cancer is present as one or more lesions in the subject. In some embodiments, the cancer is present as more than one lesion in the subject.

In certain embodiments, the one or more lesions is present in prostate tissue of the subject. In certain embodiments, more than one lesion is present in prostate tissue of the subject. In certain embodiments, more than one lesion is present in non-prostate tissue of the subject.

According to another aspect, the present invention provides a method for the detection and diagnosis of a secondary cancer related to a primary cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu;

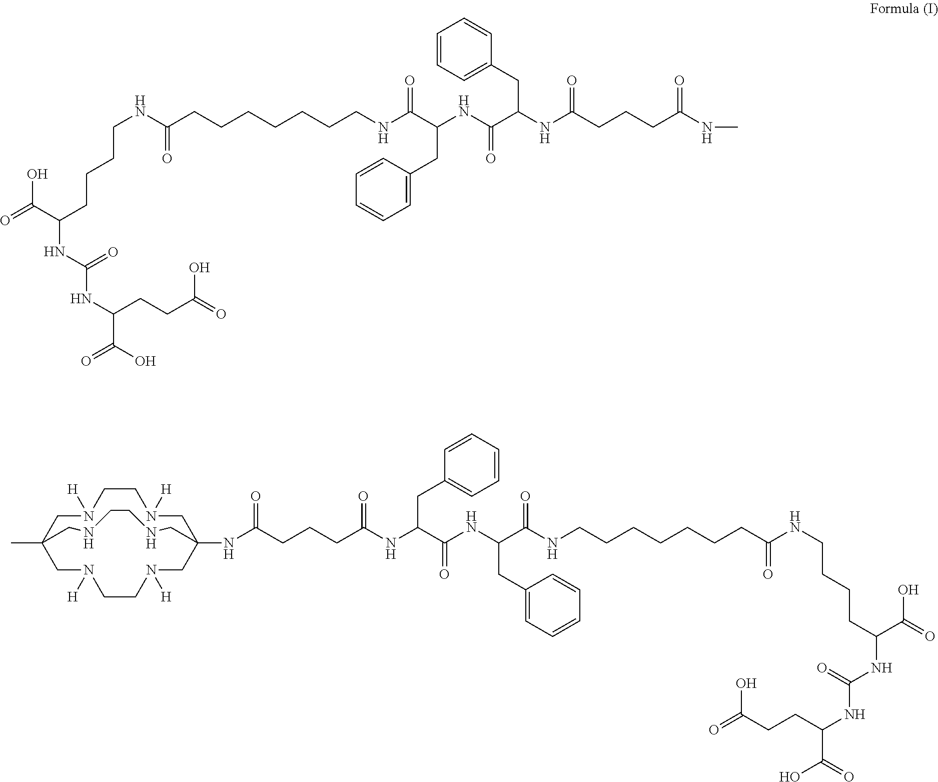

ii) imaging of the subject by PET imaging at a time of at least about 8 hours after administration of the compound of Formula (I).

According to another aspect, the present invention provides a method for the detection and diagnosis of a secondary cancer related to a primary cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu;

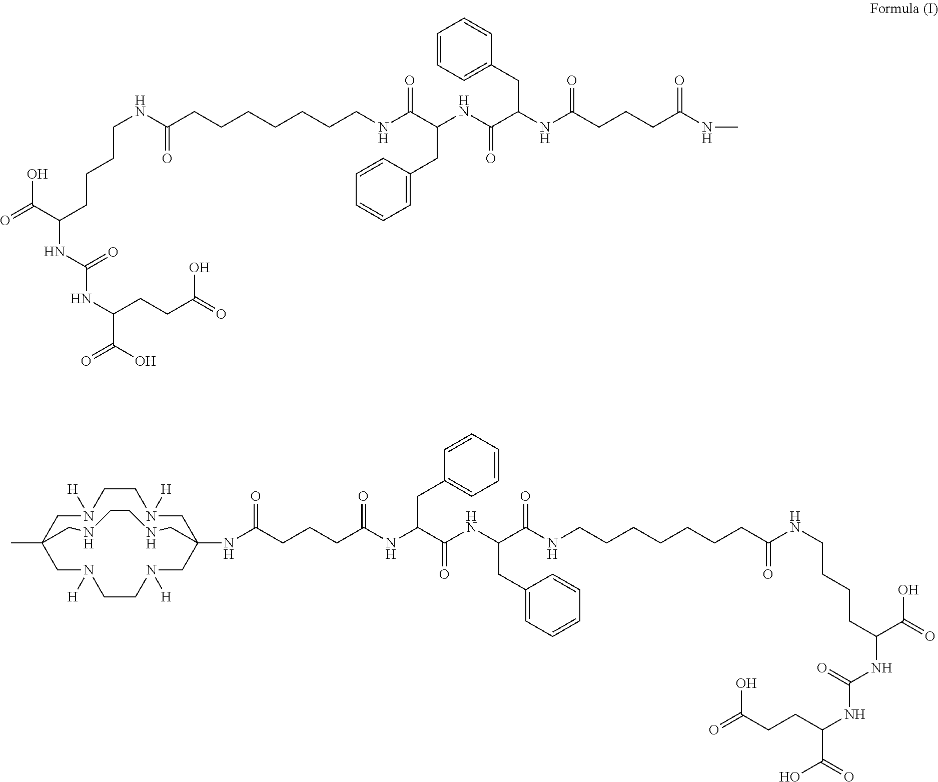

ii) imaging of the subject by PET imaging; and
iii) determining the tumor-to-background ratio (TTBR) of a lesion visualised by imaging;
   wherein the tumor-to-background ratio (TTBR) of a lesion associated with the cancer in a the subject as detected by imaging in step ii) is more than about 40.

According to another aspect, the present invention provides a method for the detection and diagnosis of a secondary cancer related to a primary cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu;

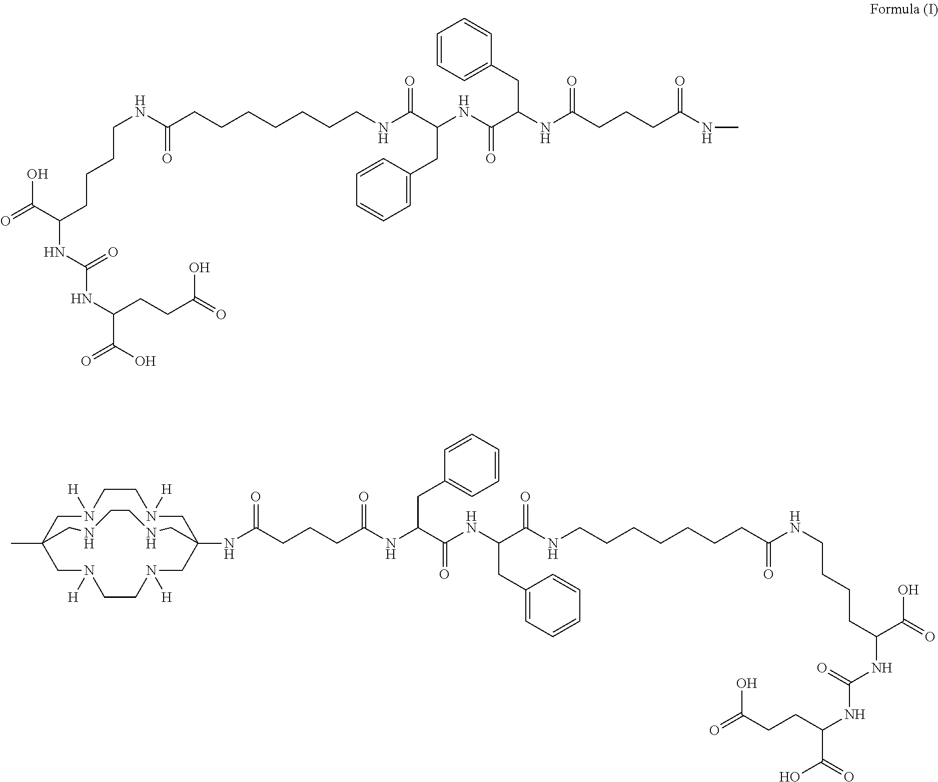

ii) imaging of the subject by PET imaging at a time of at least about 8 hours after administration of the compound of Formula (I);
iii) determining the tumor-to-background ratio (TTBR) of a lesion visualised by imaging;
   wherein the tumor-to-background ratio (TTBR) of a lesion associated with the cancer in a the subject as detected by imaging in step ii) is more than about 40.

According to another aspect, the present invention provides a method for the detection and diagnosis of a secondary cancer related to a primary cancer associated with overexpression of a PSMA receptor in a subject in need thereof, the method comprising:
i) administering to the subject a dose of between about 100 to about 300 MBq of a compound of Formula (I) or a salt thereof complexed with ⁶⁴Cu;
ii) imaging of the subject by PET imaging at least once between about 30 minutes and about 36 hours after administration of the compound of Formula (I); and
iii) diagnosing the secondary cancer on the basis of one or more images obtained in step ii),

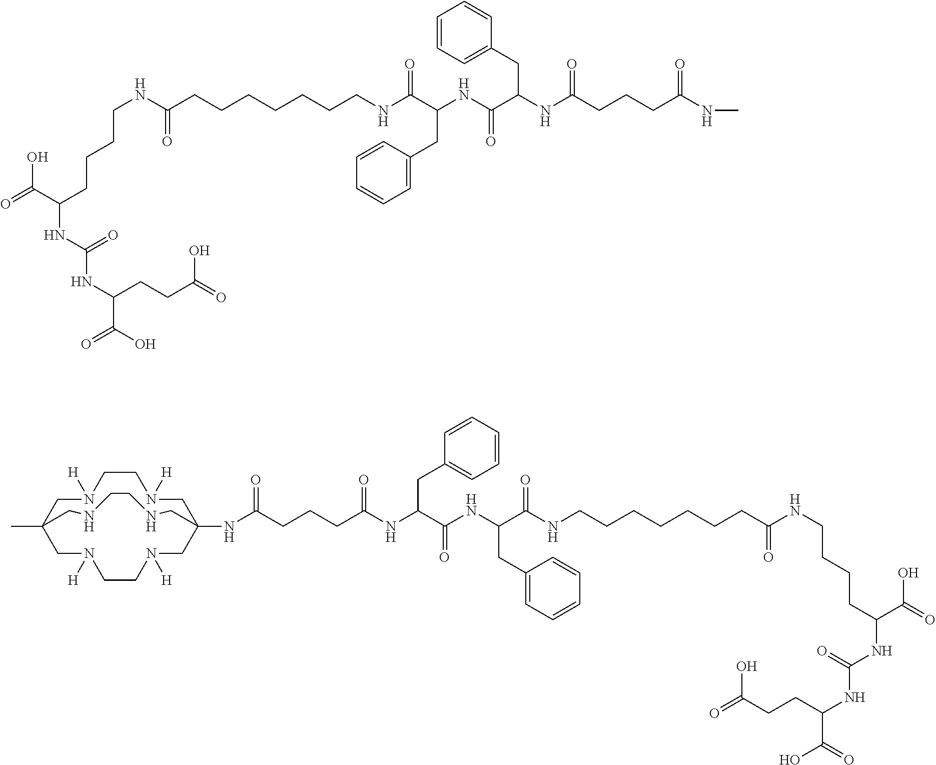

wherein the image of the cancer is at a higher resolution relative to ⁶⁸Ga-PSMA-11 administered under standard of care conditions.

The present inventors have found that the administration of the compound of Formula (I) complexed with ⁶⁴Cu and subsequent PET imaging allows for the visualisation of a secondary cancer. Secondary cancers are located at different (i.e. non-primary) sites and result from metastasis of the primary cancer. This can be seen in FIG. 3, where imaging with the compound of Formula (I) complexed with ⁶⁴Cu in accordance with the methods disclosed herein clearly identifies a lesion as seen in FIG. 3 (A), whereas imaging the same subject with the comparative ⁶⁸ Ga product does not identify the same lesion. This is seen by the absence of a signal at the same location in FIG. 3 (B). Without wishing to be bound by theory, the present inventors believe that the methods disclosed herein allow for improved detection and identification of cancers associated with the PSMA receptor. Once identified, the present methods also allow for more effective and efficient diagnosis of the cancer, as the images of the subject show greater intensity such that readers of the images can interpret and subsequently diagnose the cancer with greater certainty. FIG. 9 also shows the identification of a secondary cancer (in the superdiaphragmatic area) in a subject, where imaging after administration of an ¹⁸F-labelled ligand revealed negative/equivocal results (SUVmax=2.3) and imaging after administration of ⁶⁴Cu Sar-bisPSMA on the same day also gave negative results (SUVmax=4.3). However imaging of the same subject the following day (i.e. Day 1) clearly identified a lesion with an SUVmax value of 17.5, i.e. 4 times higher than when imaged the previous day. Without wishing to be bound by theory, the present inventors believe that the methods disclosed herein allow for next-day imaging of patients to which a dose of ⁶⁴Cu-Sar-bisPSMA has been administered. Current products and protocols for imaging and/or diagnosis of a cancer associated with a PSMA receptor do not allow for next-day imaging, due to the half-life of the radioisotope that is used, elimination of the ligand and radioisotope from the patient, leakage of the radioisotope from the ligand and general lack of stability of such ligands.

In certain embodiments, the primary cancer is a prostate cancer.

In certain embodiments, the secondary cancer is at a non-prostate site.

In certain embodiments, the secondary cancer is in a lymph node. In other embodiments, the secondary cancer is in bone, the bladder, lungs or the liver.

In certain embodiments, the secondary cancer is associated with biochemical recurrence.

The present inventors believe that the methods disclosed herein allow for the diagnosis of a secondary cancer. Since a secondary cancer may have a lower density of PSMA receptors, the increased time between administration of the ⁶⁴Cu radiotracer and imaging allows for sufficient accumulation of the radiotracer at cancer sites having a lower density of PSMA receptors such that images of a sufficient resolution are obtained. Accumulation of a greater amount of the radiotracer provides a better tumour-to-background ratio, thus leading to images of greater contrast and higher resolution.

Without wishing to be bound by theory, the present inventors believe that the use of the methods disclosed herein allow for detection and diagnosis of a secondary cancer, which is not possible when the SOC radiotracer ⁶⁸Ga-PSMA-11 radiotracer is used under comparable conditions. In some embodiments, PET imaging of a subject with prostate cancer in accordance with methods disclosed herein indicates overexpression of PSMA in one or more lymph nodes and that a secondary cancer is present in the lymph node.

In certain embodiments, the method further comprises confirmation of the secondary cancer by biopsy.

According to a further aspect, the present invention provides a method for determining the TNM stage of a prostate cancer in a subject, the method comprising
i) administering to the subject a dose of between about 100 MBq to about 300 MBq of a compound of Formula (I) or a salt thereof complexed with ⁶⁴Cu;
ii) imaging of the subject by PET imaging; and
iii) determining the TNM stage of the cancer on the basis of one or more images obtained in step ii) and the ISUP grade of the tumour;

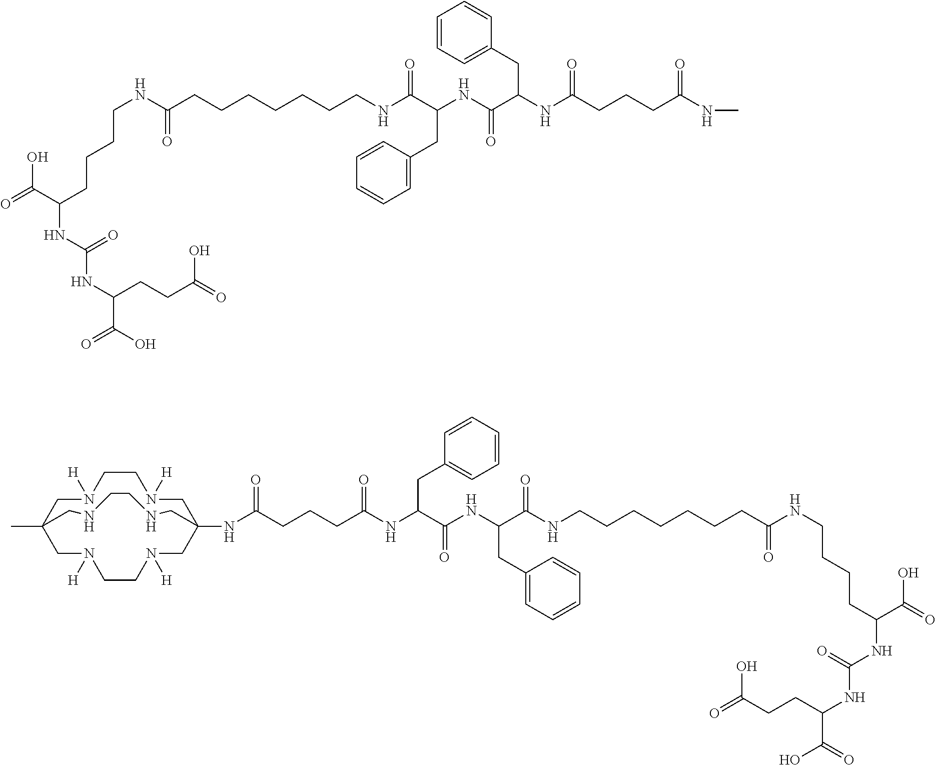

wherein the ISUP grade of the tumour is determined prior to steps i) to iii).

According to a further aspect, the present invention provides a method for determining the TNM stage of a prostate cancer in a subject, the method comprising
i) administering to the subject a dose of between about 100 MBq to about 300 MBq of a compound of Formula (I) or a salt thereof complexed with ⁶⁴Cu;
ii) imaging of the subject by PET imaging at a time of at least about 8 hours after administration of the compound of Formula (I); and
iii) determining the TNM stage of the cancer on the basis of one or more images obtained in step ii) and the ISUP grade of the tumour;

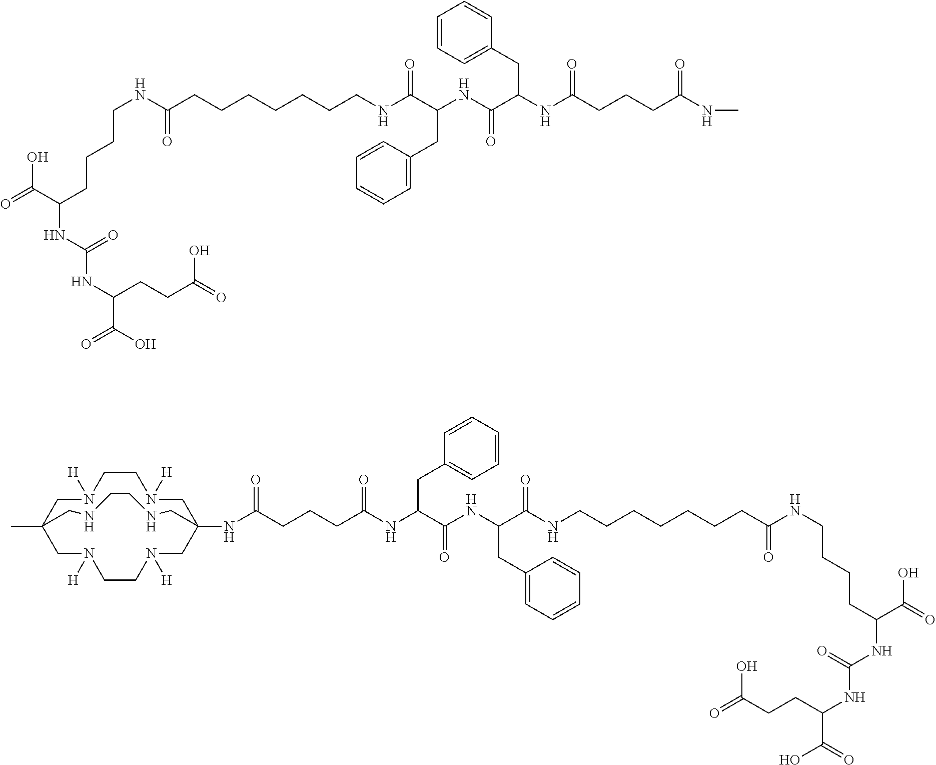

wherein the ISUP grade of the tumour is determined prior to steps i) to iii).

According to a further aspect, the present invention provides a method for determining the TNM stage of a prostate cancer in a subject, the method comprising
i) administering to the subject a dose of between about 100 MBq to about 300 MBq of a compound of Formula (I) or a salt thereof complexed with ⁶⁴Cu;
ii) imaging of the subject by PET imaging at least once between about 30 minutes and about 36 hours after administration of the compound of Formula (I); and
iii) determining the stage of the cancer on the basis of one or more images obtained in step ii) and the ISUP grade of the tumour;

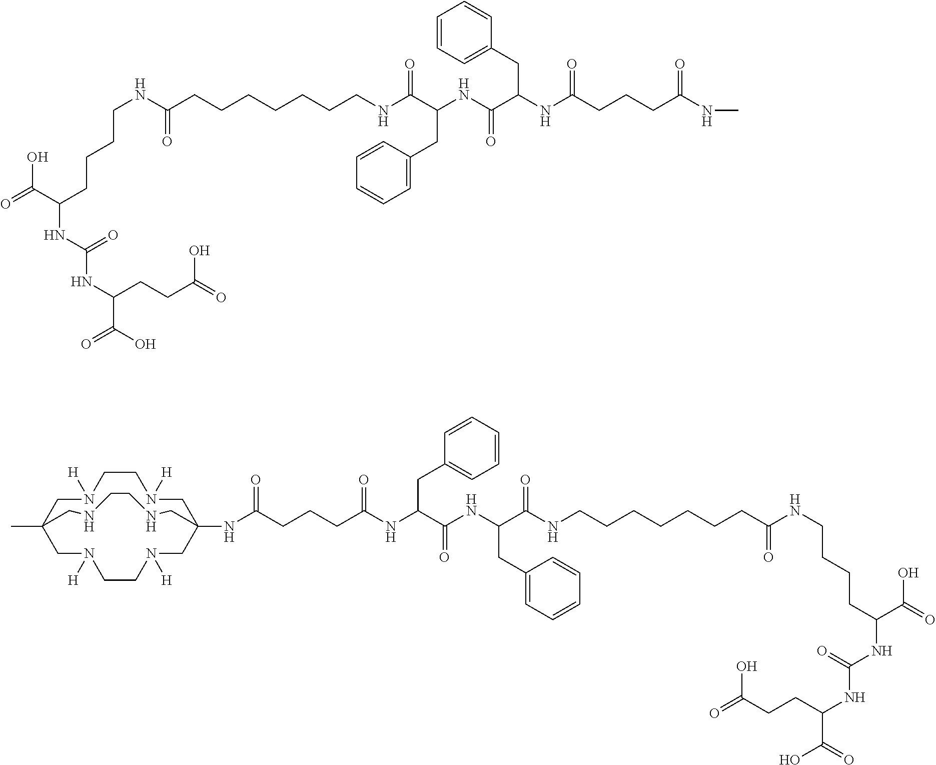

wherein the ISUP grade of the tumour is determined prior to steps i) to iii).

In certain embodiments, imaging of the subject by PET imaging occurs about 12 hours after administration of the compound of Formula (I) complexed with ⁶⁴Cu. In other embodiments, imaging of the subject by PET imaging occurs about 18 hours after administration of the compound of Formula (I) complexed with ⁶⁴Cu. In other embodiments, imaging of the subject by PET imaging occurs about 24 hours after administration of the compound of Formula (I) complexed with ⁶⁴Cu.

In certain embodiments, the ISUP grade of the tumour is Grade 1, Grade 2, Grade 3, Grade 4 or Grade 5.

In certain embodiments, the ISUP grade of the tumour is determined by calculation of the Gleason Score. In certain embodiments, the Gleason Score is 6, 7, 8, 9 or 10.

In certain embodiments, the TNM stage of the prostate cancer is selected from one or more of TX, T0, T1, T2, T3, T4, NX, N0, N1, N2, N3, MX, M0 or M1.

According to another aspect, the present invention provides a method for re-evaluating the TNM stage of a prostate cancer in a subject, the method comprising:
i) administering to the subject a dose of between about 100 MBq to about 300 MBq of a compound of Formula (I) or a salt thereof complexed with ⁶⁴Cu;

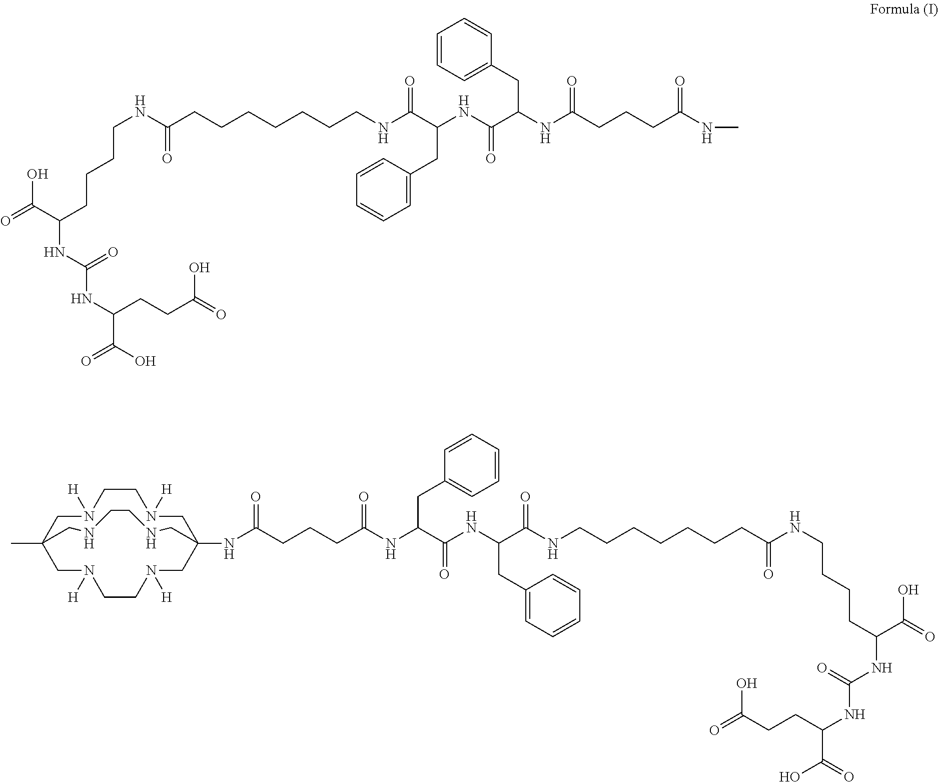

ii) imaging of the subject by PET imaging;
iii) first determining the stage of the prostate cancer on the basis of one or more images obtained in step ii); and
iv) repeating steps i) and ii) and determining the stage of the prostate cancer on the basis of one or more images obtained by imaging of the subject after the second administration of the compound of Formula (I) in accordance with step i).

In certain embodiments, re-evaluation of the TNM stage of a prostate cancer in a subject is performed in order to determine progression of the cancer in the subject.

According to a further aspect, the present invention provides a method for determining the progression of a cancer associated with the overexpression of a PSMA receptor in a subject, the method comprising:
i) administering to the subject a dose of between about 100 MBq to about 300 MBq of a compound of Formula (I) or a salt thereof complexed with ⁶⁴Cu;

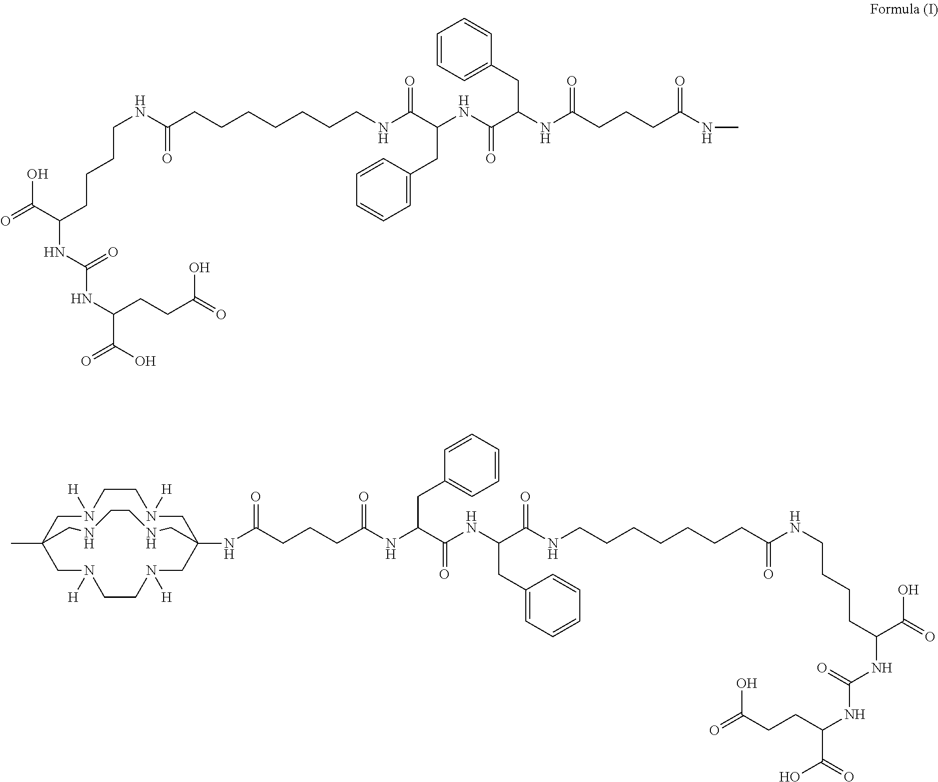

ii) imaging of the subject by PET imaging;
iii) first determining the TNM stage of the prostate cancer on the basis of one or more images obtained in step ii); and
iv) repeating steps i) and ii) and determining the TNM stage of the prostate cancer on the basis of one or more images obtained by imaging of the subject after the second administration of the compound of Formula (I) in accordance with step i).

According to another aspect, the present invention provides a method for re-evaluating the TNM stage of a prostate cancer in a subject, the method comprising:
i) administering to the subject a dose of between about 100 MBq to about 300 MBq of a compound of Formula (I) or a salt thereof complexed with ⁶⁴Cu;

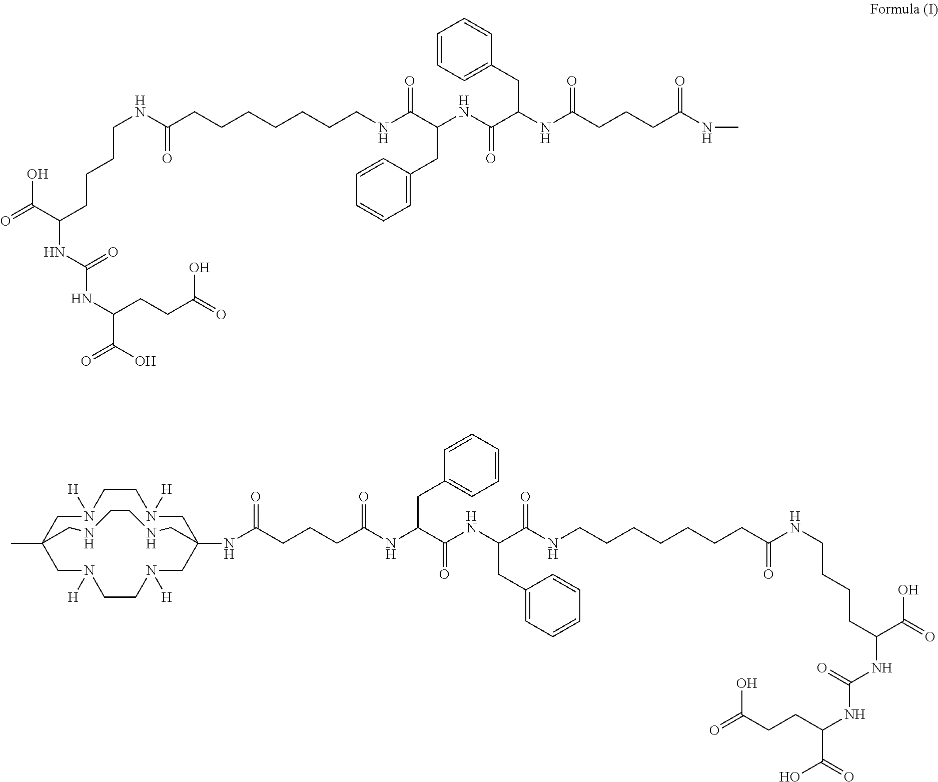

ii) imaging of the subject by PET imaging at least once between about 30 minutes and about 36 hours after administration of the compound of Formula (I);
iii) first determining the stage of the prostate cancer on the basis of one or more images obtained in step ii); and
iv) repeating steps i) and ii) and determining the stage of the prostate cancer on the basis of one or more images after the second administration in accordance with step i).

In certain embodiments, the TNM stage of the prostate cancer after re-evaluating is higher.

In certain embodiments, the TNM stage of the prostate cancer after re-evaluating includes a N1, N2 or N3 stage.

In certain embodiments, the TNM stage of the prostate cancer after re-evaluating is lower.

In certain embodiments, the TNM stage of the prostate cancer after re-evaluating includes a T0, N0 or M0 stage.

In a further embodiment, the prostate cancer is PSMA-expressing metastatic castrate resistant prostate cancer (mCRPC) or is progressive mCRPC despite prior androgen deprivation therapy and at least either enzalutamide and/or abiraterone (or other such androgen receptor pathway inhibitors). In another embodiment, the prostate cancer is characterised by biochemical resistance in the subject.

### BRIEF DESCRIPTION OF FIGURES

FIG. 1. Ex vivo tumor uptake expressed as percentage injected activitiy per gram of tissue (% lAg) (mean±/−SEM, n=3/group) in LNCap tumor bearing NSG mice following injection of either monomeric [⁶⁴Cu]Cu-SarPSMA (2 MBq, 0.9 nmol of peptide) or dimeric [⁶⁴Cu]Cu-Sar-bisPSMA (2 MBq, 0.2 nmol of peptide). At 1 hour after administration, uptake of ⁶⁴Cu-Sar-bisPSMA is approximately double the uptake of the monomeric ⁶⁴Cu-Sar-PSMA compound. At 6 and 24 hours after administration, the amount of ⁶⁴Cu-Sar-bisPSMA that is retained at the tumor site is far greater than the amount of ⁶⁴Cu-SarPSMA that is retained under the same conditions.

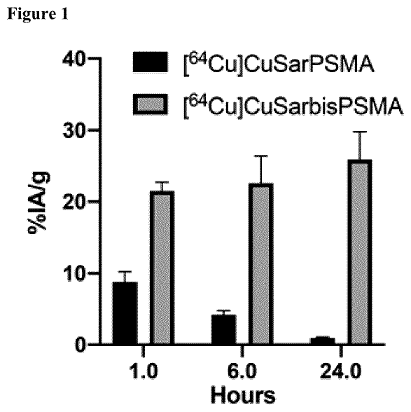

FIG. 2. Intra-individual comparison of PET/CT images obtained after administration of ⁶⁸Ga-PSMA-11 (1A and 1C) and ⁶⁴Cu-Sar-bisPSMA (1B and 1D) showing clearer delineation of lesions and higher SUVmax.

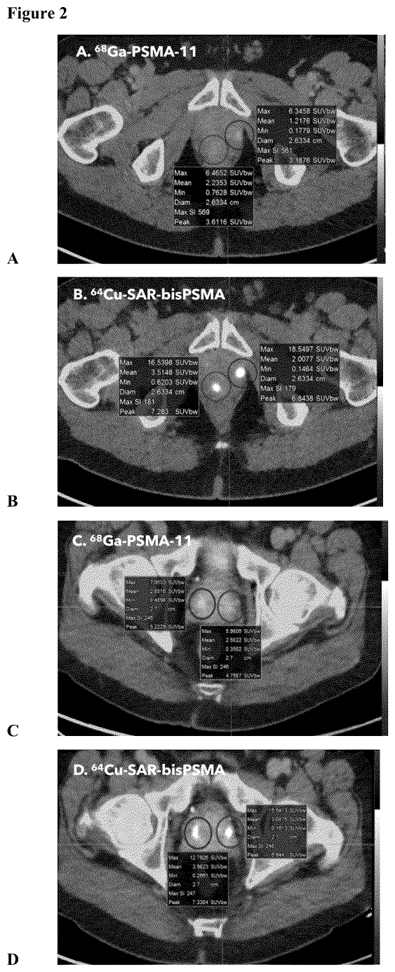

FIG. 3. Detection of a secondary lesion in the lymph node of a subject by PET/CT after administration of ⁶⁴Cu-Sar-bisPSMA as seen in FIG. 2A. Comparative image of the same subject after administration of ⁶⁸Ga-PSMA-11 is shown in FIG. 2B, where uptake was not detected.

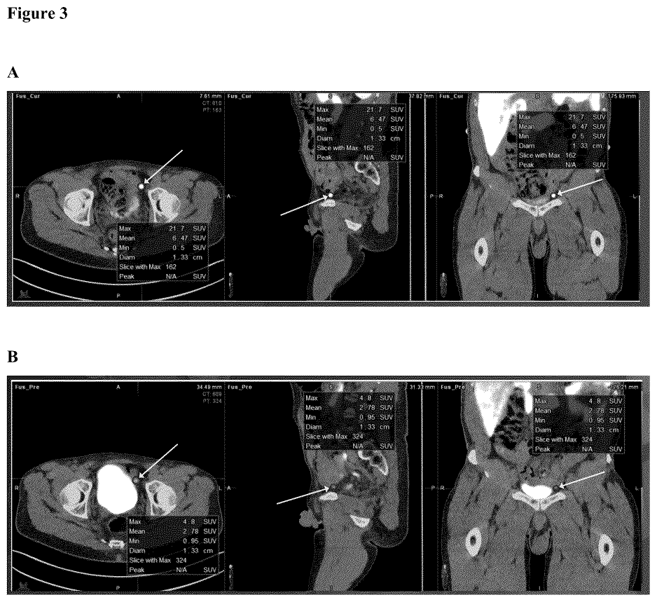

FIG. 4. SUVmax of concordant lesions as detected by PET/CT after administration of ⁶⁴Cu-Sarbis-PSMA and ⁶⁸Ga-PSMA-11, as detected by (A) Reader 1 and (B) Reader 2. Both readers determined that lesions detected after administration of ⁶⁴Cu-SarbisPSMA showed a higher SUVmax than the same lesions after administration of ⁶⁸Ga-PSMA-11.

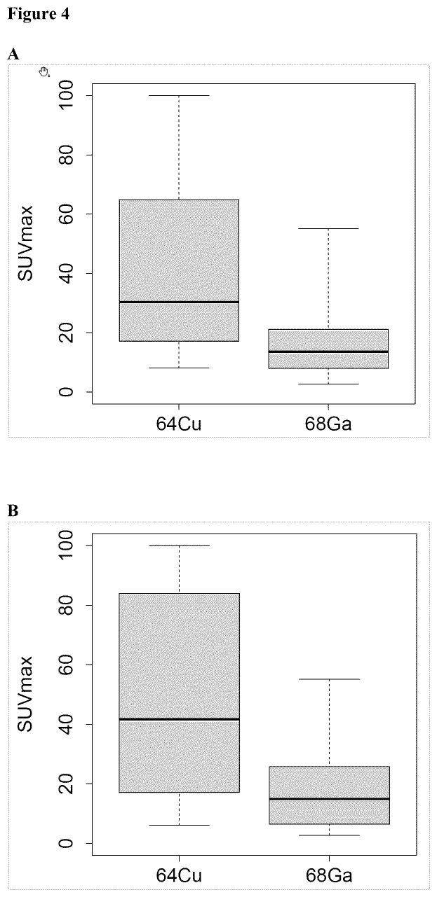

FIG. 5. SUVmean of concordant lesions as detected by PET/CT after administration of ⁶⁴Cu-Sar-bisPSMA and ⁶⁸Ga-PSMA-11, as detected by (A) Reader 1 and (B) Reader 2. Both readers determined that lesions detected after administration of ⁶⁴Cu-Sar-bis-PSMA showed a higher SUVmean than the same lesions after administration of ⁶⁸Ga-PSMA-11.

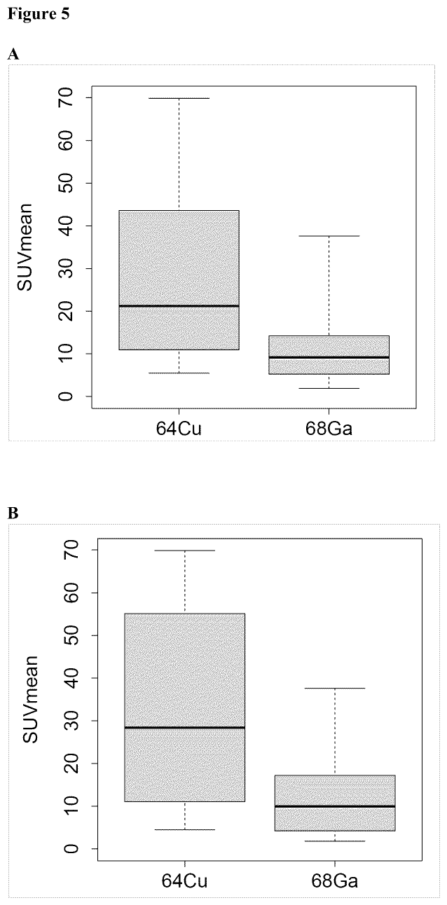

FIG. 6. The tumor to background ratio of concordant lesions as detected by PET/CT after administration of ⁶⁴Cu-Sar-bisPSMA and ⁶⁸Ga-PSMA-11, as detected by (A) Reader 1 and (B) Reader 2. Both readers determined that lesions detected after administration of ⁶⁴Cu-SarbisPSMA showed a higher tumour to background ratio.

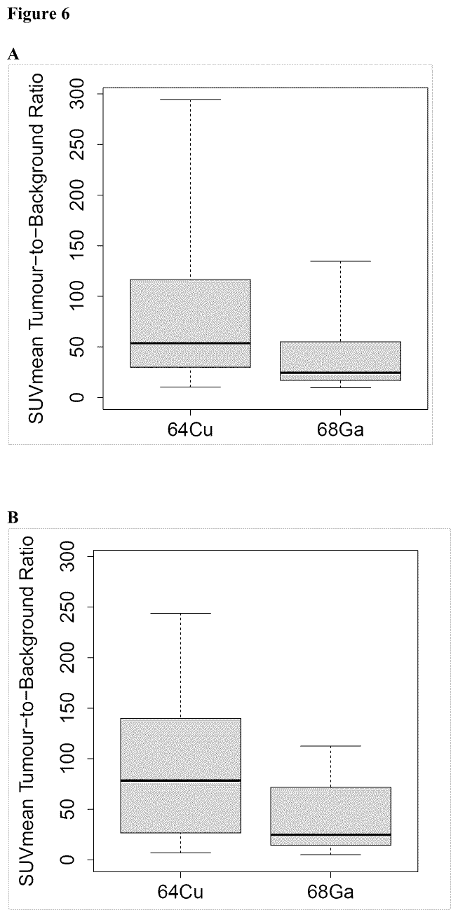

FIG. 7. ⁶⁴Cu-SAR-bisPSMA PET showing a positive pelvic lymph node (LN; red circle, maximum intensity projection). CT-guided needle biopsy of the lesion was performed and was negative for PC. This was followed by an excisional biopsy of the lesion, which confirmed the presence of PC by histopathology. Next-day imaging (FIG. 8B) showed an increase of SUVmax (maximum standard uptake value) of more than double compared to same-day imaging (FIG. 8A), from 20.8 on Day 0 to 50.4 on Day 1.

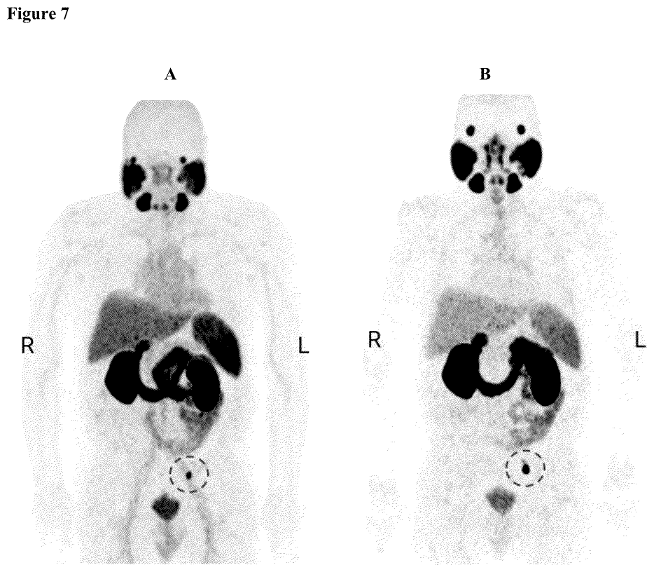

FIG. 8. Next-day images (FIG. 8B, i.e. images taken the day after administration of ⁶⁴Cu-Sar-bisPSMA) of the superdiaphragmatic region of a subject to which ⁶⁴Cu-Sar-bisPSMA was administered. The lesion in this region of the subject was identified by three readers upon imaging of the subject the day following administration of ⁶⁴Cu-Sar-bisPSMA, whereas the same lesion was less apparent on the basis of same-day imaging (FIG. 8A).

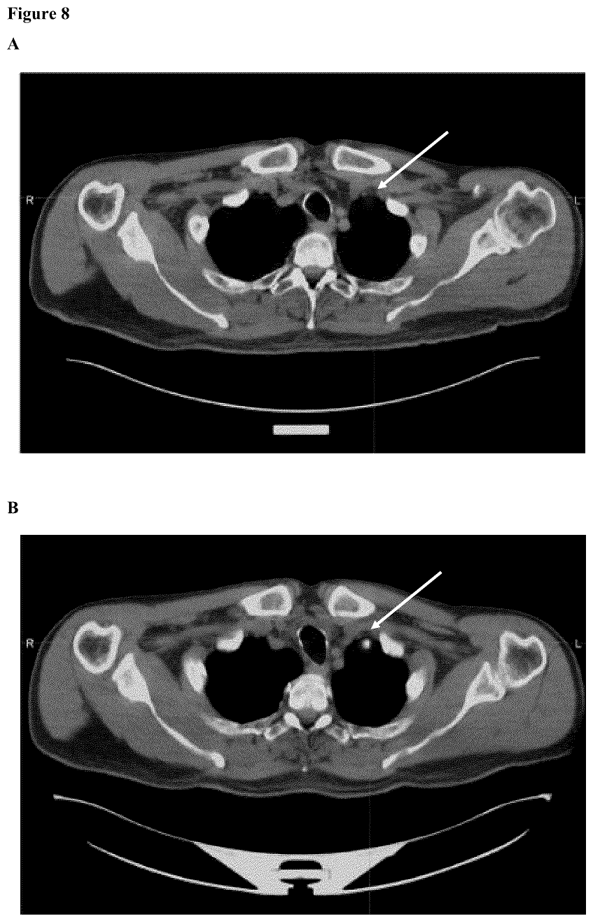

FIG. 9. PET/CT fusion images of a subject to which ¹⁸F-DCYPyL was administered as SOC (FIG. 9A, SUVmax=2.3), showing negative/equivocal screening results. Same-day (FIG. 9B, SUVmax=4.3) and next-day (FIG. 9C, SUVmax=17.5) imaging of the same subject to which ⁶⁴Cu-Sar-bisPSMA was administered. A lesion in the pelvic region was identified by next-day imaging after administration of ⁶⁴Cu-Sar-bisPSMA, where the same lesion was not visualised after same-day imaging of the subject after administration of ⁶⁴Cu-Sar-bisPSMA.

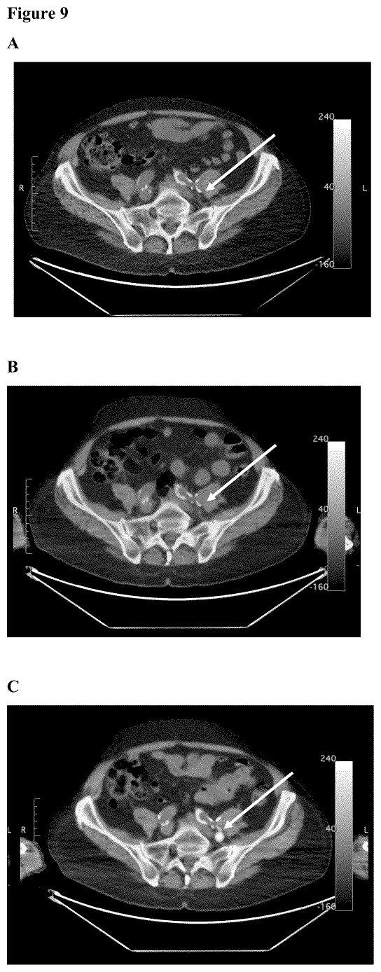

FIG. 10. Administration of ⁶⁴Cu-Sar-bisPSMA to a patient and PET imaging of the pelvic, extra-pelvic (retroperitoneal) and prostatic bed regions on the same day (FIG. 10A) and the following day (FIG. 10B). As seen in FIG. 10B, imaging of the patient the following day after administration of ⁶⁴Cu-Sar-bisPSMA revealed more positive lesions and more clearly visualised lesions initially detected with same day imaging as seen in FIG. 10A.

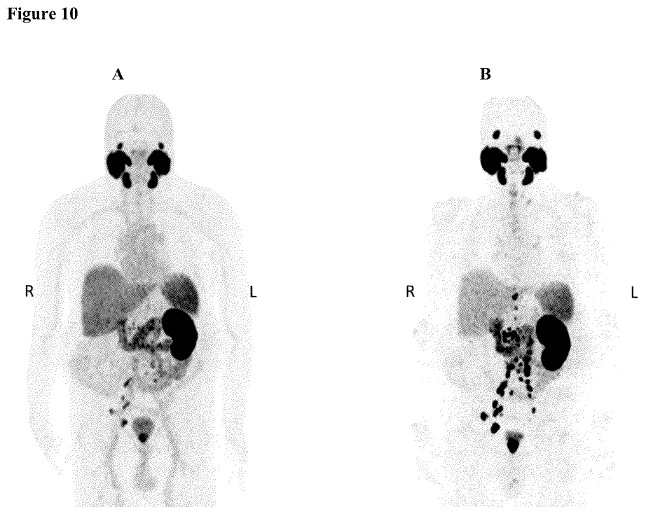

FIG. 11. Administration of ⁶⁴Cu-Sar-bisPSMA to a patient and PET imaging of the pelvic, extra-pelvic (retroperitoneal) and prostatic bed regions on the same day (FIG. 11A) and the following day (FIG. 11B). Both images show a lesion in the pelvic bone, with pelvic lymph nodes only visualised with imaging on the following day. The average SUVmax for the bone and pelvic lymph node lesions increased from 9.8 (FIG. 11A) to 20.0 (FIG. 11B). A prostatic bed lesion was only visualised with imaging on the following day, as seen in FIG. 11B.

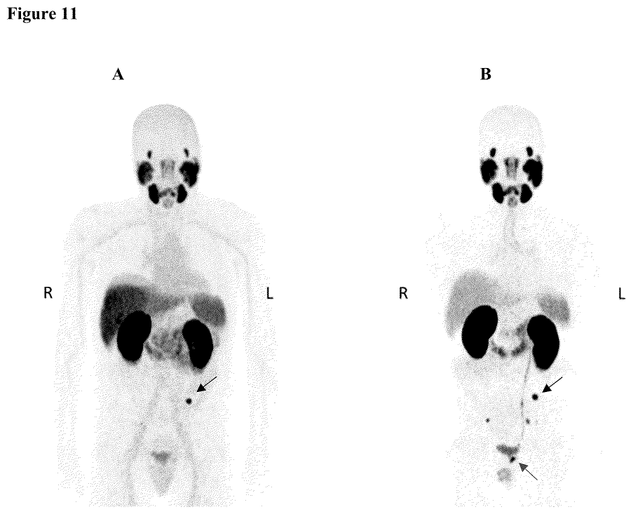

FIG. 12. Administration of ⁶⁴Cu-Sar-bisPSMA and subsequent PET imaging of the pelvic, extra-pelvic (retroperitoneal) and prostatic bed regions of the subject on the day of administration (FIG. 12A) and the following day (FIG. 12B). Imaging on the following delay showed additional positive lesions in the subject, compared to same day imaging.

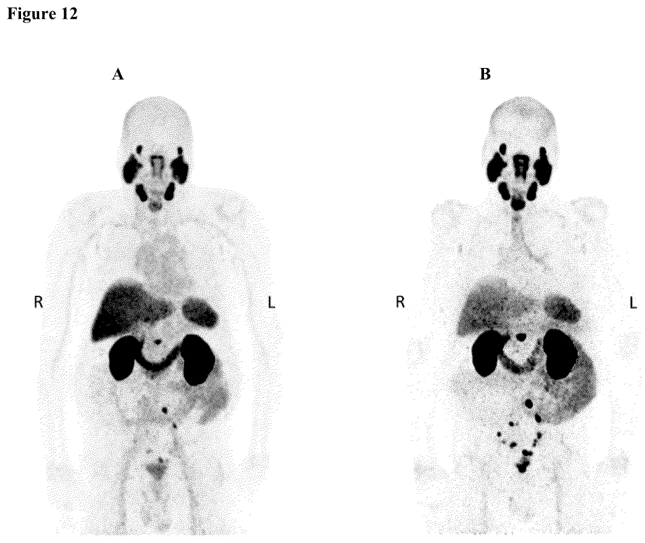

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “about” or “approximately” as used herein means within an acceptable error range for the particular value as determined by one of ordinary skill in the art, which will depend in part on how the value is measured or determined, i.e., the limitations of the measurement system.

Unless defined otherwise, all technical and scientific terms used herein have the same meaning as commonly understood by those of ordinary skill in the art to which the invention belongs. For the purposes of the present invention, the following terms are defined below.

The compound of Formula (I) as defined herein is a sarcophagine appended with two lysine-urea-glutamate motifs via linkers and has the following structure:

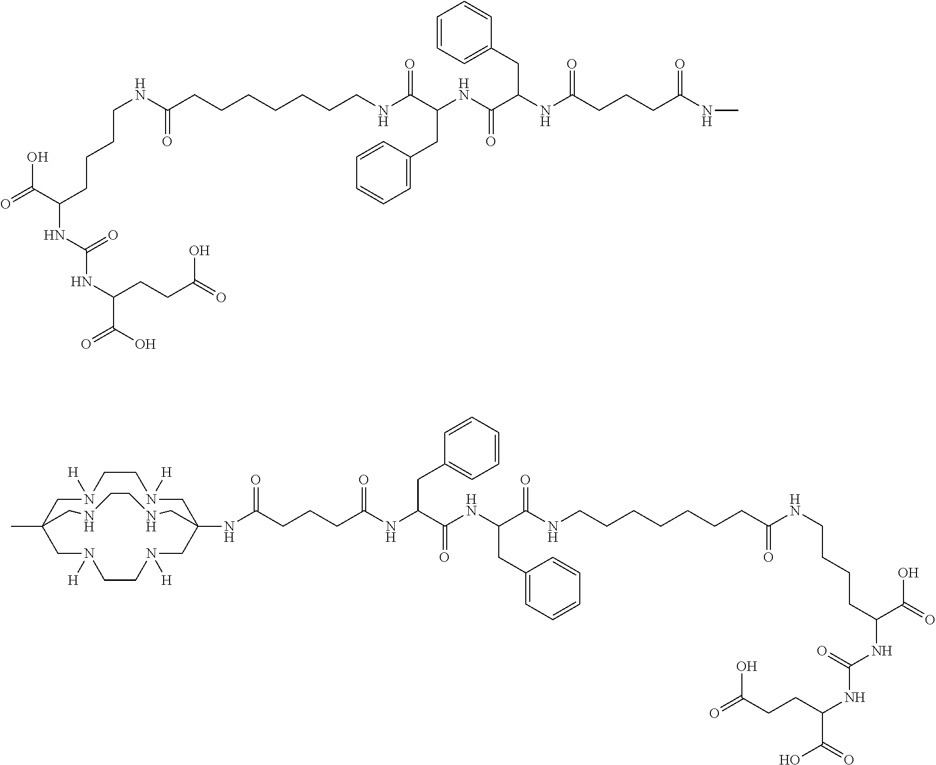

The compound of Formula (I) is also known as Sar-bisPSMA.

In certain embodiments, the compound of Formula (I) has the following structure:

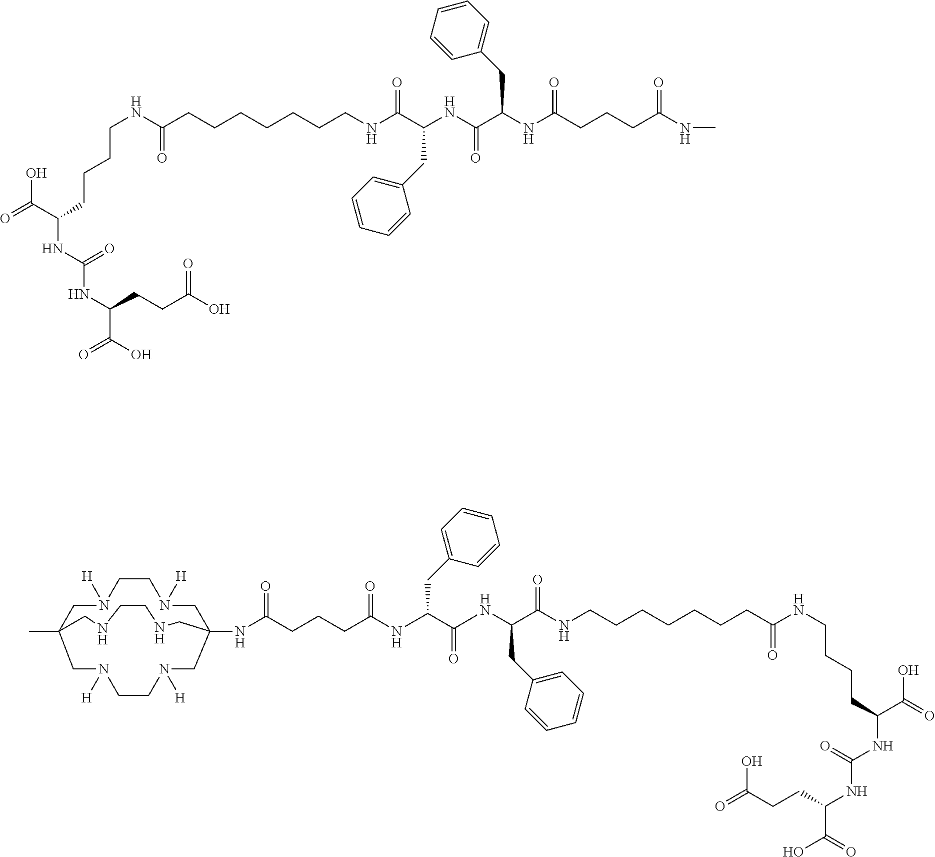

As used herein, the compound ⁶⁸Ga-PSMA-11 refers to a ⁶⁸Ga radioisotope complexed to the compound PSMA-11 having the following structure:

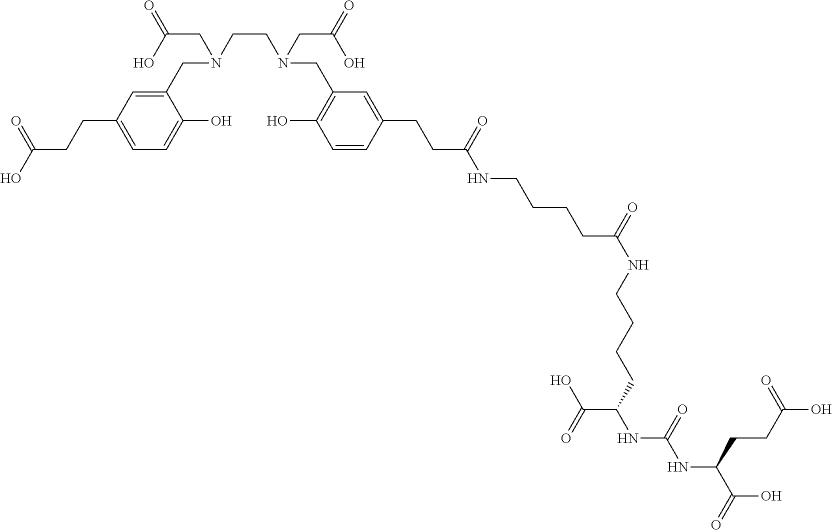 [SMILES: O=C(O)CCc1ccc(O)c(CN(CCN(CC(=O)O)Cc2cc(CCC(=O)NCCCCC(=O)NCCCC[C@H](NC(=O)N[C@@H](CCC(=O)O)C(=O)O)C(=O)O)ccc2O)CC(=O)O)c1]

PSMA-11 complexed with a ⁶⁸Ga radioisotope is also known as HBED-CC, HBED, PSMA-HBED or Prostamedix™.

As used herein, the compound ¹⁸F-DCFPyL (¹⁸F piflufolastat) refers to the compound DCFPyL complexed with a ¹⁸F radioisotope having the following structure:

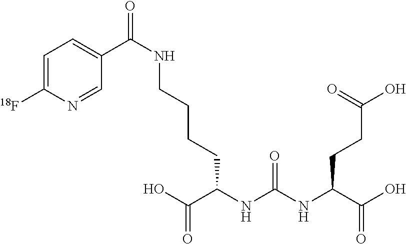 [SMILES: O=C(O)CC[C@H](NC(=O)N[C@@H](CCCCNC(=O)c1ccc([18F])nc1)C(=O)O)C(=O)O]

The term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic acids. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of a compound with the appropriate inorganic or organic acid via any of a number of known methods. Alternatively, alkali and alkaline earth metal salts can be prepared by reacting a compound with the appropriate base via a variety of known methods. The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention and specified formulae.

As used herein, the term “cancer” broadly encompasses neoplastic diseases characterised by abnormal cell growth with the potential to invade or spread to other parts of the body. The cancer may be benign, which does not spread to other parts of the body. The cancer may be malignant, meaning that the cancer cells can spread through the circulatory system or lymphatic system. The term as used herein includes all malignant, i.e. cancerous, disease states. The cancer may be present as a tumour. Accordingly, the term “tumour” is used generally to define any malignant cancerous or pre-cancerous cell growth, and may include leukemias, but is particularly directed to solid tumours or carcinomas such as melanomas, colon, lung, ovarian, skin, breast, pancreas, pharynx, brain, prostate, CNS, and renal cancers (as well as other cancers).

In an embodiment, the condition is breast cancer, colon cancer, lung cancer, ovarian cancer, prostate cancer, head and/or neck cancer, or renal, gastric, pancreatic cancer, brain cancer a hematologic malignancy such as lymphoma or leukaemia

As used herein the term “standard-of-care” refers to a treatment that is the best treatment known for the condition. For prostate cancer, the current standard-of-care includes the administration of a dose of ⁶⁸Ga-PSMA-11 with radioactivity between 111 MBq and 259 MBq (in accordance with the FDA), with images obtained by PET imaging for the purposes of diagnosis after a time of about 1 hour.

As used herein, the term “detection” refers to the visualisation of a disease in a patient. The detection of a disease may involve the administration of an agent to the patient and subjecting the patient to one or more imaging modalities, where one or more images taken of the patient is processed so as to display the localisation of the agent in the body of the patient. The process of imaging for the purposes of detection may also include steps such as imaging of the patient in order to determine a baseline image, an anatomical image or the like, in order to allow for a comparison to be made with images produced after administration of an agent.

As used herein, the term “diagnosis” refers to the detection and subsequent identification of a disease in a patient. The diagnosis of a disease may take into account one or more signs and symptoms displayed by the patient and may include one or more examination or tests, such as imaging, blood tests or biopsies. For example, a disease may be diagnosed in a patient on the basis of signs and symptoms reported by the patient and results from one or more imaging modalities, biopsies of tissue and blood tests to detect specific markers of disease. In certain embodiments, the methods disclosed herein are used to diagnose a cancer in a patient, where the methods include the use of PET imaging, biopsy and/or blood tests of the patient.

As used herein, the term “prognosis” refers to the prediction of the course of a disease in a patient that has been diagnosed with a particular disease. For example, where a patient has been diagnosed with a particular disease, the information obtained in the process of diagnosis may be used to evaluate the extent or severity of disease, which may in turn be used to form a prognosis specific to the patient. In certain embodiments, the methods disclosed herein allow for the imaging of one or more cancers in a subject, where the imaging of the cancer provides information in relation to its location, severity and the like, such that a prognosis or possible course or outcome of the disease can be made.

As used herein, the term “resolution” in relation to an image refers more specifically to “spatial resolution” and is related to the distance between measurements. In relation to the images obtained in accordance with methods disclosed herein, images of higher resolution have more detail. In images of higher resolution, the distance between independent measurements is lower than in images of lower resolution. More specifically, the term “spatial resolution” in PET imaging refers to differentiation of two objects. PET imaging that allows for higher resolution imaging means that two objects that are closer together are able to be differentiated, whereas the same objects under conditions of lower resolution may not result in differentiation.

Resolution in PET imaging may be compared by quantifying and comparing values such as a standardized uptake value (SUV). Different statistical measures of the SUV may also be determined, for example, the mean (SUVmean), median, maximum (SUVmax) and the like.

As used herein, the term “tumour to background ratio” (TTBR) is the ratio between uptake of radiation in a tumour or lesion associated with a cancer and remaining radiation in the background after administration of the radiotracer. For example, the TTBR may be defined as the ratio of radiation uptake in a region of interest and radiation in a suitable background region.

In particular embodiments, the TTBR of a tumor or lesion may be more than about 40, about 45, about 50, about 55, about 60, about 65, about 70, about 75, about 80, about 85, about 90, about 95, about 100, about 105, about 110, about 115, about 120, about 125, about 130, about 135, about 145 or about 150. In some embodiments, the TTBR may be in a range between of two of the aforementioned values. In some embodiments, the TTBR is in a range of between about 40 and about 150. In some embodiments, the TTBR is in a range of between about 40 and about 140. In some embodiments, the TTBR is in a range of between about 40 and about 130. In some embodiments, the TTBR is in a range of between about 40 and about 120. In some embodiments, the TTBR is in a range of between about 40 and about 110. In some embodiments, the TTBR is in a range of between about 40 and about 100. In some embodiments, the TTBR is in a range of between about 40 and about 90. In some embodiments, the TTBR is in a range of between about 40 and about 80. In some embodiments, the TTBR is in a range of between about 40 and about 70.

As used herein, the term “primary cancer” refers to a cancer that is the original or first tumour present in the patient. In certain embodiments of the methods disclosed herein, the cancer is a primary cancer. In further embodiments, the cancer is a primary cancer and is a cancer of the prostate. In some embodiments, there may be more than one primary cancer, where one of the primary cancers is a prostate cancer.

As used herein, the term “secondary cancer” refers to a cancer that is derived from cells of the primary cancer that have spread to a new location, i.e. where the primary cancer has metastasised. Where metastasis has occurred and has formed a secondary cancer, this secondary cancer is the same type of cancer as the primary cancer. In certain embodiments, the secondary cancer is a prostate cancer.

Where a cancer metastasises, cells from the primary cancer leave the primary site, spread to a new location and form a new tumour at the new location. In certain embodiments, the new location a secondary cancer is a lymph node. Lymph nodes form part of the lymphatic system and are involved in drainage of lymph fluid from organs and other parts of the body. Since lymph nodes are found throughout the body and act as a passage to the blood stream and circulatory system, cells that leave a primary tumour site typically contact a lymph node first. This means that secondary cancers are often found in lymph nodes and lymph tissue.

In certain embodiments, the secondary cancer is at a non-prostate site. In some embodiments of the present invention, the secondary cancer is found in a lymph node. In other embodiments, the secondary cancer is found in bone. In other embodiments, the secondary cancer is found in the bladder. In other embodiments, the secondary cancer is found in the lungs. In other embodiments, the secondary cancer is found in the liver.

As used herein, the “TNM staging system” refers to a particular framework for describing and classifying the stages of a cancer. The TNM system takes into account information relating to the primary tumour (T), the number of nearby lymph nodes with cancer (N) and whether the cancer has metastasised (M) and spread from the primary tumour to other parts of the body.

TNM Staging System

| | | |
|---|---|---|
| T - size and the | TX | Main tumor cannot be measured |
| extent of the main | T0 | Main tumor cannot be found |
| (i.e. primary) tumor | T1, | Refers to the size and/or extent of the main tumor, i.e. the higher |
| | T2, | the number after the T, the larger the tumor or the more it has |
| | T3, | grown into nearby tissues. T's may be further divided to provide |
| | T4 | more detail, such as T3a and T3b |
| N - number of | NX | Cancer in nearby lymph nodes cannot be measured |
| regional lymph | N0 | No cancer in nearby lymph nodes |
| nodes having | N1, | Refers to the number and location of lymph nodes that contain |
| cancer | N2, | cancer, i.e. the higher the number after the N, the more lymph |
| | N3 | nodes that contain cancer |
| M - extent of distant | MX | Metastasis cannot be measured |
| metastasis | M0 | Cancer has not spread to other parts of the body |
| | M1 | Cancer has spread to other parts of the body |

As used herein, the “Gleason Score” refers to a tumour staging system used to grade prostate cancer in a patient. Cancerous cells fall into 5 distinct patterns as they change from normal prostate cells to tumour cells and are subsequently graded on a scale of 1 to 5. The Gleason Score takes into account the types of cells identified by histopathology present in a biopsy sample from a patient. A first Gleason grade is assigned to the most predominant pattern of cells in the sample and a second Gleason grade is assigned to the second most predominant pattern. The two grades are then added together to give the Gleason Score, which theoretically ranges from 2 to 10, however pathologists typically assign scores of between 6 and 10.

As used herein the term “ISUP Grade” refers to the grade assigned to a prostate cancer in accordance with the guidelines approved by the International Society of Urological Pathology (ISUP). The ISUP Grade is based on the Gleason Score as described above, i.e. the ISUP Grade also relies on a biopsy sample taken from the patient.

| ISUP Grade | Gleason Score | Definition |
|---|---|---|
| Grade 1 | 2 to 6 | Only individual discrete well-formed glands |
| Grade 2 | 3 + 4 = 7 | Predominantly well-formed glands with lesser component |
| | | of poorly formed/fused/cribiform glands |
| Grade 3 | 4 + 3 = 7 | Predominantly poorly formed/fused/cribiform glands with |
| | | lesser component of well-formed glands |
| Grade 4 | 4 + 4 = 8 | Only poorly formed/fused/cribriform glands |
| | 3 + 5 = 8 | Predominantly well-formed glands and lesser component |
| | | lacking glands (or with necrosis) |
| | 5 + 3 = 8 | Predominantly lacking glands (or with necrosis) and lesser |
| | | component of well-formed glands |
| Grade 5 | 9 to 10 | Lacking gland formation (or with necrosis) with or without |
| | | poorly formed/fused/cribriform glands |

As used herein, the term “prostate specific antigen” (PSA) refers to a glycoprotein enzyme that is secreted by the epithelial cells of the prostate gland. Although PSA is produced by both normal and malignant prostate cells, PSA levels are often elevated in subjects with prostate cancer. In certain embodiments, the PSA level of a subject may be between about 4 ng/ml and about 10 ng/ml. In other embodiments, the PSA level of a subject may be less than about 4 ng/ml. In other embodiments, the PSA level of a subject may be more than about 10 ng/ml. While an elevated PSA level often indicates that a patient has prostate cancer, some patients with PSA levels less than about 4 ng/ml have prostate cancer, while other patients with higher levels between about 4 ng/ml and about 10 ng/mL do not have prostate cancer. Determination of the PSA level in a subject may be used in conjunction with other techniques, e.g. PET imaging, CT imaging and the like as disclosed herein, to arrive at a diagnosis of prostate cancer.

As used herein, the term “biochemical recurrence” in relation to prostate cancer relates to the suspected recurrence of prostate cancer on the basis of rising levels of PSA after either radical prostatectomy or radiation therapy, cryotherapy or brachytherapy. In certain embodiments, biochemical recurrence of prostate cancer after radical prostatectomy is characterised by detectable or rising levels of PSA that is ≥0.2 ng/ml with a confirmatory PSA ≥0.2 ng/ml (as per American Urological Association recommendation). In other embodiments, biochemical recurrence of prostate cancer after radiation therapy, cryotherapy, or brachytherapy is characterised by an increase in PSA level that is elevated by ≥2 ng/ml above the nadir (as per American Society for Therapeutic Radiology and Oncology-Phoenix consensus definition).

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

In certain embodiments, the subject is a male patient.

The compounds are, however, typically used in the form of pharmaceutical compositions which are formulated depending on the desired mode of administration. The compositions are prepared in manners well known in the art.

The invention in other embodiments provides a pharmaceutical pack or kit comprising one or more containers filled with one or more of the ingredients of the pharmaceutical compositions of the invention. In such a pack or kit can be found at least one container having a unit dosage of the agent(s). Conveniently, in the kits, single dosages can be provided in sterile vials so that the clinician can employ the vials directly, where the vials will have the desired amount and concentration of compound and radio nucleotide which may be admixed prior to use. Associated with such container(s) can be various written materials such as instructions for use, or a notice in the form prescribed by a governmental agency regulating the manufacture, use or sale of pharmaceuticals, imaging agents or biological products, which notice reflects approval by the agency of manufacture, use or sale for human administration.

The compound of Formula (I) complexed with ⁶⁴Cu is provided as a composition for parenteral injection and may comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin. Other additives may include formulation stabilisers such as sodium gentisate, sodium ascorbate and the like.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

In the methods disclosed herein, the compound of Formula (I) complexed with ⁶⁴Cu is administered as an aqueous formulation, where the formulation is administered parenterally, preferably intravenously. In certain embodiments, the compound of Formula (I) complexed with ⁶⁴Cu is administered intravenously as a bolus or infusion. In some embodiments, the compound of Formula (I) complexed with ⁶⁴Cu is administered as a single bolus intravenous injection.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

The following examples are illustrative of the disclosure and should not be construed as limiting in any way the general nature of the disclosure of the description throughout this specification.

The compound of Formula (I) complexed with ⁶⁴Cu is supplied to clinical sites as a ready-to-use sterile formulation for intravenous injection. The final formulation of the compound of Formula (I) complexed with ⁶⁴Cu contains sodium phosphate buffer, sodium gentisate and sodium ascorbate and has a final pH in the range of 4 to 8. ⁶⁸Ga-PSMA-11 is produced at each clinical site as required.

### Example 1—PET Imaging of Patients

Patients with untreated, histopathology-proven, intermediate- to high-risk PC, planned for radical prostatectomy underwent ⁶⁸Ga-PSMA-11 PET/CT per institutional practice and were subsequently allocated to 1 out of 3 dose cohorts (1:1:3) to receive either 100 MBq, 150 MBq or 200 MBq of ⁶⁴Cu-SAR-bisPSMA, respectively. The specified dose of ⁶⁴Cu-Sar-bisPSMA was administered to each patient as a single bolus IV injection. Alternatively, a specified dose of either ⁶⁸Ga-PSMA-11 or ¹⁸F-DCFPyL was administered to the patient. A PET/CT scan was acquired at 3 hours (+1 hour) post-injection of ⁶⁴Cu-SAR-bisPSMA, ⁶⁸Ga-PSMA-11 or ¹⁸F-DCFPyL. PET/CT scans at other times after injection of the compound were acquired as required.

### Example 2—Acquisition Parameters for PET Imaging

Patients were scanned using the standard clinical whole-body PET/CT acquisition protocol defined for each clinical site and in accordance with the Image Acquisition Manual, which contains details of scan acquisition, processing parameters and quality assurance procedures. The same scanner was used for each visit. Typical imaging acquisition parameters are below:

| PARAMETERS* | VALUES |
|---|---|
| CT Energy | [symbol] 30 kVp |
| CT Effective [symbol] | [symbol] (with automated exposure control) |
| CT slice thickness acquisition and reconstruction | SOC (2-3 mm without gap) |
| CT Pitch | 0.8 to 1 |
| CT [symbol] | medium smooth |
| PET Bed position Whole Body | 6 to 8 |
| PET Time per Bed position | 180 [symbol] |
| PET Reconstruction Images | CT-based [symbol] Correction (AC) |
| | [symbol] Correction (SC) |
| | resolution recovery |
| | [symbol] |
| PET Reconstruction parameters | iteration and subsets: [symbol] -specific |
| | [symbol] size as far standard [symbol] scans |
| | Past-Reconstruction FIlter: [symbol] 6 to 8 mm |

[symbol]

indicates data missing or illegible when filed

### Example 3—Analysis of PET Images

Two independent, blinded, central readers assessed the detection of primary PC on the 200 MBq ⁶⁴Cu-SAR-bisPSMA PET/CTs as the primary endpoint. To compare the intensity of lesions on ⁶⁸Ga-PSMA-11 and ⁶⁴Cu-SAR-bisPSMA PET, both readers determined the SUVs (SUVmax, SUVmean) and tumour-to-background ratio (ratio of lesion SUVmax and background SUVmean) in up to 5 concordant lesions between ⁶⁸Ga-PSMA-11 and ⁶⁴Cu-SAR-bisPSMA PET/CT as an exploratory endpoint.

Tables 1 and 2 below compare SUV parameters obtained from PET/CT images after administration of ⁶⁸Ga-PSMA-11 and a dose of 200 MBq of ⁶⁴Cu-SAR-bisPSMA from each reader. Imaging with ⁶⁴Cu-SAR-bisPSMA showed consistently higher SUVmax and SUVmean values when compared to images obtained with ⁶⁸Ga-PSMA-11.

TABLE 1

| | | | | | | | | Median | p- |
|---|---|---|---|---|---|---|---|---|---|
| Parameter | Imaging | N | Mean | SD | Median | Min | Max | Difference | value*^{#} |
| SUVₘₐₓ | ⁶⁴Cu | 17 | 40.28 | 31.54 | 31.40 | 8 | 100 | 14.85 | p < 0.001 |
| | ⁶⁸Ga | 17 | 15.80 | 13.98 | 10.08 | 2.7 | 48.8 | | |
| SUVₘₑₐₙ | ⁶⁴Cu | 17 | 27.25 | 21.71 | 21.76 | 5.5 | 69.9 | 9.64 | p = 0.0047 |
| | ⁶⁸Ga | 17 | 10.57 | 9.57 | 6.58 | 1.8 | 33.3 | | |
| TTBR | ⁶⁴Cu | 17 | 77.25 | 68.33 | 49.07 | 10.3 | 227.3 | 27.15 | p = 0.0015 |
| | ⁶⁸Ga | 17 | 35.90 | 29.42 | 21.91 | 9.6 | 106.4 | | |

TTBR = Tumour-to-Background Ratio.
*Comparison of imaging methods undertaken with two-sided Wilcoxon signed-rank test;
^{#}Violation of normality assumption confirmed with Shapiro-Wilk Test for Normality (p < 0.05). The lesions were averaged for each participant so that each participant contributes once to the summary statistics.

TABLE 2

| | | | | | | | | Median | p- |
|---|---|---|---|---|---|---|---|---|---|
| Parameter | Imaging | N | Mean | SD | Median | Min | Max | Difference | value* |
| SUVₘₐₓ | ⁶⁴Cu | 13 | 47.37 | 33.58 | 41.00 | 6.1 | 100 | 27.03 | p < 0.001 |
| | ⁶⁸Ga | 13 | 18.19 | 15.29 | 14.58 | 2.7 | 48.8 | | |
| SUVₘₑₐₙ | ⁶⁴Cu | 13 | 31.78 | 23.35 | 28.07 | 4.4 | 69.9 | 18.44 | p < 0.001 |
| | ⁶⁸Ga | 13 | 12.21 | 10.40 | 9.79 | 1.8 | 33.1 | | |
| TTBR | ⁶⁴Cu | 13 | 86.40 | 69.51 | 76.34 | 6.7 | 212.8 | 45.37 | p < 0.001 |
| | ⁶⁸Ga | 13 | 36.96 | 31.75 | 23.90 | 5 | 95.6 | | |

TTBR = Tumour-to-Background Ratio.
*Comparison of imaging methods undertaken with two-sided Wilcoxon signed-rank test;
^{#} Violation of normality assumption confirmed with Shapiro-Wilk Test for Normality (p < 0.05). The lesions were averaged for each participant so that each participant contributes once to the summary statistics.

Tables 3 and 4 below compare SUV parameters obtained from PET/CT images after administration of ⁶⁸Ga-PSMA-11 (200 MBq) and ⁶⁴Cu-SAR-bisPSMA (200 MBq) from each reader.

TABLE 3

| | | | | | | | | Median | p- |
|---|---|---|---|---|---|---|---|---|---|
| Parameter | Imaging | N | Mean | SD | Median | Min | Max | Difference | value*^{#} |
| SUVₘₐₓ | ⁶⁴Cu | 28 | 41.66 | 30.27 | 30.26 | 8.00 | 100.00 | 14.23 | p < 0.001 |
| | ⁶⁸Ga | 28 | 17.45 | 13.71 | 13.53 | 2.70 | 55.10 | | |
| SUVₘₑₐₙ | ⁶⁴Cu | 28 | 27.41 | 20.93 | 21.20 | 5.40 | 69.90 | 9.26 | p < 0.001 |
| | ⁶⁸Ga | 28 | 11.71 | 9.37 | 9.12 | 1.80 | 37.60 | | |
| TTBR | ⁶⁴Cu | 28 | 83.38 | 72.13 | 53.55 | 10.30 | 294.10 | 27.94 | p < 0.001 |
| | ⁶⁸Ga | 28 | 40.04 | 31.54 | 24.29 | 9.60 | 134.40 | | |

TTBR = Tumour-to-Background Ratio.
*Comparison of imaging methods undertaken with two-sided Wilcoxon signed-rank test;
^{#}Violation of normality assumption confirmed with Shapiro-Wilk Test for Normality (p < 0.05). The lesions were averaged for each participant so that each participant contributes once to the summary statistics.

TABLE 4

| | | | | | | | | Median | p- |
|---|---|---|---|---|---|---|---|---|---|
| Parameter | Imaging | N | Mean | SD | Median | Min | Max | Difference | value* |
| SUVₘₐₓ | ⁶⁴Cu | 16 | 49.73 | 34.53 | 41.66 | 6.1 | 100 | 27.99 | p < 0.001 |
| | ⁶⁸Ga | 16 | 19.82 | 16.93 | 14.93 | 2.7 | 55.1 | | |
| SUVₘₑₐₙ | ⁶⁴Cu | 16 | 33.24 | 23.75 | 28.4 | 4.4 | 69.9 | 18.78 | p < 0.001 |
| | ⁶⁸Ga | 16 | 13.29 | 11.56 | 9.94 | 1.8 | 37.6 | | |
| TTBR | ⁶⁴Cu | 16 | 93.86 | 76.09 | 78.37 | 6.7 | 243.9 | 46.93 | p < 0.001 |
| | ⁶⁸Ga | 16 | 41.61 | 35.53 | 24.69 | 5 | 112.4 | | |

TTBR = Tumour-to-Background Ratio.
*Comparison of imaging methods undertaken with two-sided Wilcoxon signed-rank test;
^{#} Violation of normality assumption confirmed with Shapiro-Wilk Test for Normality (p < 0.05). The lesions were averaged for each participant so that each participant contributes once to the summary statistics.

Table 5 compares the number of positive, negative and indeterminate images as assessed by each reader. Reader 1 detected primary prostate cancer in 100% of patients to whom 200 MBq of ⁶⁴Cu-Sar-bisPSMA was administered, while Reader 2 detected 85.7% of patients. This is in contrast to 77.8% and 83.3%, respectively, when ⁶⁸Ga-PSMA-11 was administered.

TABLE 5

| | ⁶⁴Cu-SAR-bisPSMA PET | | | ⁶⁸Ga-PSMA-11 PET | | |
|---|---|---|---|---|---|---|
| Reader | Positive | Negative | Indeterminate | Positive | Negative | Indeterminate |
| 1 | 18/18 | 0/18 | 0/18 | 14/18 | 0/18 | 4/18 |
| 2 | 12/14* | 0/14* | 2/14* | 15/18 | 0/18 | 3/18 |

*4 scans were excluded by the reader deeming them non-evaluable

The resulting True Positive Rate (TPR) and False Negative Rate (FNR) were similar for both ⁶⁴Cu-Sar-bisPSMA and ⁶⁸Ga-PSMA-11, as seen in Table 6.

TABLE 6

| | ⁶⁴Cu-SAR-bisPSMA PET | | ⁶⁸ Ga-PSMA-11 PET | | |
|---|---|---|---|---|---|
| | % TPR | % FNR | % TPR | % FNR¹ | |
| Reader | (95% CI) | (95% CI) | (95% CI) | (95% CI) | P-value² |
| 1 | 100.0 | 0.0 | 77.8 | 22.2 | 0.13 |
| | (81.5; 100.0) | (0.0; 18.5) | (52.4; 93.6) | (6.4; 47.6) | |
| 2 | 85.7 | 14.3 | 83.3 | 16.7 | 1.0 |
| | (57.2; 98.2) | (1.8; 42.8) | (58.6; 96.4) | (3.6; 41.4) | |

¹Indeterminate results were analysed as negative
²McNemar's Chi-squared test with continuity correction

The number of lesions detected by each reader with imaging by either ⁶⁴Cu-Sar-bisPSMA or ⁶⁸Ga-PSMA-11 was evaluated. These results are shown in Table 7. For both readers, the total number of lesions, the mean and median number of lesions detected as a result of imaging by ⁶⁴Cu-Sar-bisPSMA was higher when compared with imaging by ⁶⁸Ga-PSMA-11, with the difference in these values being statistically significant.

TABLE 7

| | | | | | | | | |
|---|---|---|---|---|---|---|---|---|
| | | Total # | Mean | | Median | | | |
| | | of Lesions | Number of | | Number of | | | |
| | | Detected | Lesions | Standard | Lesions | | | |
| | | (all | per | Deviation | per | Mean | Median | P- |
| Reader | PET | participants) | Participant | (SD) | Participant | Difference | Difference | value** |

All Cohorts

| | | | | | | | | |
|---|---|---|---|---|---|---|---|---|
| Reader 1 | ⁶⁴Cu | 63 | 2.2 | 1.2 | 2 | 0.41 | 0.0 | 0.1005 |
| (N = 29) | ⁶⁸Ga | 51 | 1.8 | 1.3 | 1 | | | |
| Reader 2 | ⁶⁴Cu | 34 | 2 | 1.4 | 4 | 0.52 | 0.0 | 0.0748 |
| (N = 17*) | ⁶⁸Ga | 25 | 1.5 | 0.9 | 1 | | | |

200 MBq Cohort

| | | | | | | | | |
|---|---|---|---|---|---|---|---|---|
| Reader 1 | ⁶⁴Cu | 41 | 2.3 | 1.4 | 2 | 0.28 | 0.0 | 0.4427 |
| (N = 29) | ⁶⁸Ga | 36 | 2.0 | 1.1 | 1 | | | |
| Reader 2 | ⁶⁴Cu | 31 | 2.2 | 1.5 | 2 | 0.64 | 0.0 | 0.0749 |
| (N = 17*) | ⁶⁸Ga | 22 | 1.6 | 1.0 | 1 | | | |

**Comparison of imaging methods undertaken with two-sided Wilcoxon signed-rank test. Violation of normality assumption confirmed with Shapiro-Wilk Test for Normality p < 0.05). Only participants who had evaluable scans for both imaging modalities were included in the analysis. The difference between imaging modalities is the number of lesions detected with ⁶⁴Cu-SAR-bisPSMA PET/CT relative to ⁶⁸Ga-PSMA-11 PET/CT within a participant (i.e. ⁶⁴Cu − ⁶⁸Ga). A negative value indicates more lesions detected with ⁶⁸Ga-PSMA-11 PET/CT than ⁶⁴Cu-SAR-bisPSMA PET/CT. The trial was not powered to detect differences at an individual level.

As seen in Table 8, the specificity in the detection across all readers of the PET images was high, regardless of whether images were obtained on the day of or the day following administration of ⁶⁴Cu-Sar-bisPSMA. The relative decrease in specificity with imaging on the day following administration was related to the challenges in obtaining the reference standard for the increased number of lesions identified, where biopsy of all lesions was not feasible and due to the low sensitivity of imaging with existing protocols.

TABLE 8

| | Day 0 | Day 1 |
|---|---|---|
| | Range across | Range across |
| | readers in % | readers in % |
| | (95% CI) | (95% CI) |
| CDR (patient-level) | 21.4-28.6 (10.3-44.6) | 28.6-38.1 (15.7-54.4) |
| DR (patient-level) | 44-58 (30-71) | 58-80 (43.2-90) |
| PPV (region-level) | 39.1-44.8 (19.7-64.3) | 32.7-43.3 (20.3-62.6) |
| Specificity (pelvic LNs) | 93.8-96.9 (79.2-99.9) | 81.3-87.9 (63.6-96.6) |

CDR: correct detection rate. DR: Detection rate. PPV: positive predictive value. LNs: lymph nodes. CI: confidence interval.

1. A method for the detection and/or diagnosis of a cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu; 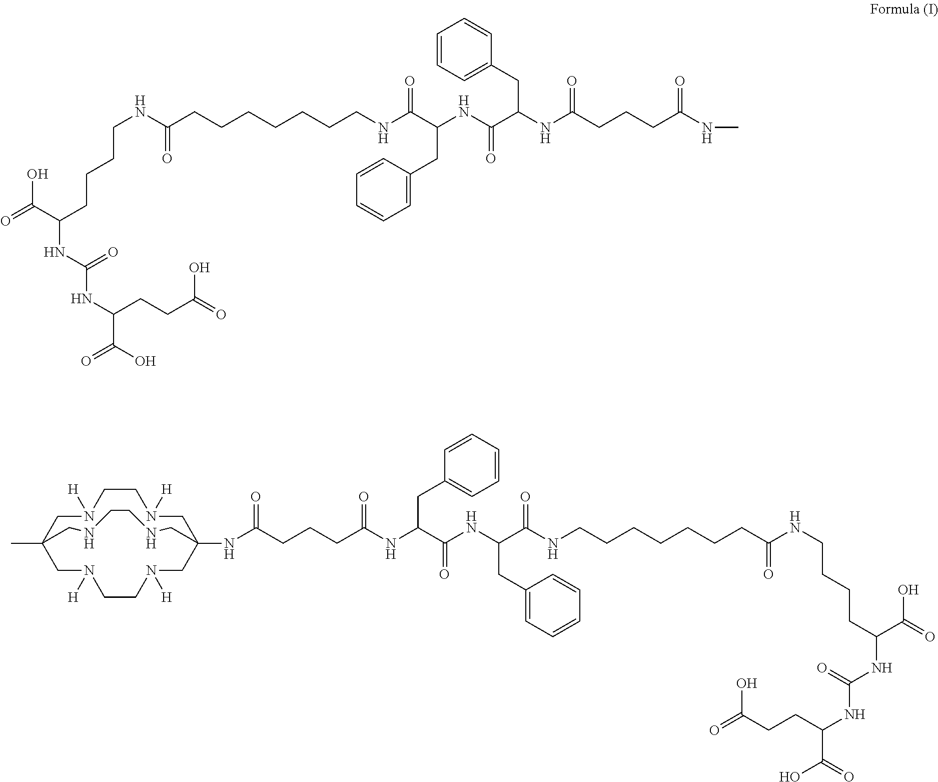
ii) imaging of the subject by PET imaging at a time of at least about 8 hours after administration of the compound of Formula (I).

2. The method according to claim 1, wherein the dose of the compound of Formula (I) or a pharmaceutically acceptable salt thereof complexed with ⁶⁴Cu is about 200 MBq.

3. The method according to claim 1, wherein the imaging of the subject by PET imaging is at a time of about 8 hours after administration of the compound of Formula (I).

4. The method according to claim 1, wherein the imaging of the subject by PET imaging is at a time of about 10 hours, about 12 hours, about 16 hours, about 20 hours, about 24 hours, about 28 hours, about 32 hours or about 36 hours after administration of the compound of Formula (I).

5. (canceled)

6. The method according to claim 1, wherein the cancer associated with the PSMA receptor is a prostate cancer.

7. The method according to claim 6, wherein the prostate cancer is associated with biochemical recurrence.

8.-51. (canceled)

52. A method for the detection and/or diagnosis of a secondary cancer related to a primary cancer associated with overexpression of a PSMA receptor in a subject in need thereof, the method comprising:
i) administering to the subject a dose of between about 100 MBq to about 300 MBq of a compound of Formula (I) or a salt thereof complexed with ⁶⁴Cu; 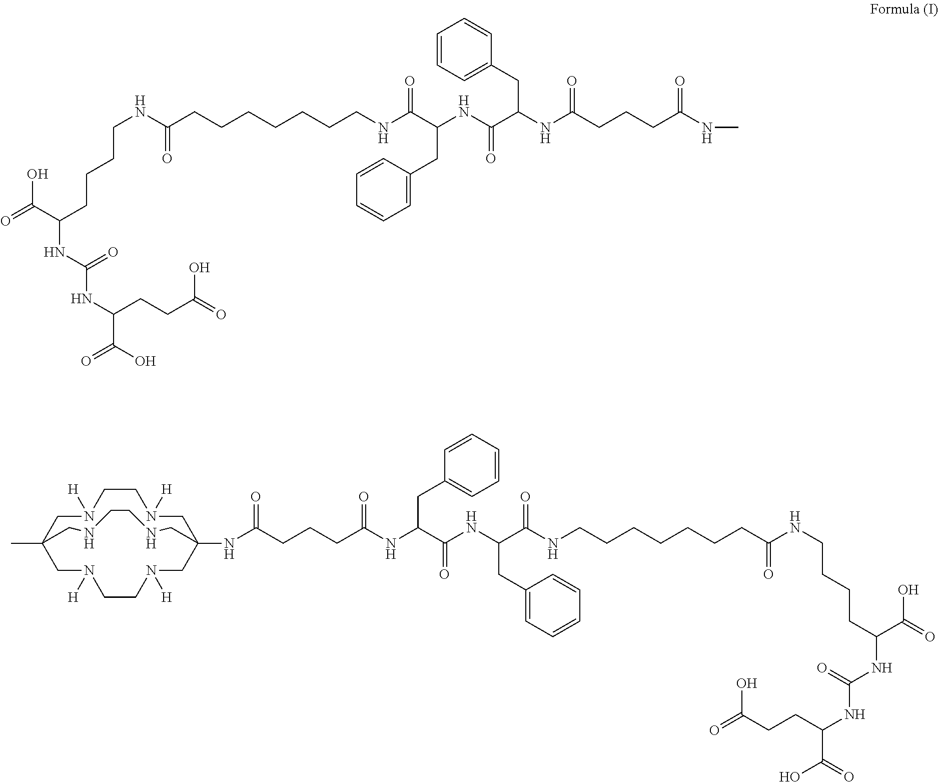
ii) imaging of the subject by PET imaging at a time of at least about 8 hours after administration of the compound of Formula (I).

53. The method according to claim 52, wherein the dose of the compound of Formula (I) or a pharmaceutically acceptable salt thereof complexed with ⁶⁴Cu is about 200 MBq.

54. The method according to claim 52, wherein the imaging of the subject by PET imaging is at a time of about 8 hours, about 10 hours, about 12 hours, about 16 hours, about 20 hours, about 24 hours, about 28 hours, about 32 hours or about 36 hours after administration of the compound of Formula (I).

55. (canceled)

56. The method according to claim 52, wherein the cancer associated with the PSMA receptor is a prostate cancer.

57. The method according to claim 56, wherein the prostate cancer is associated with biochemical recurrence.

58.-77. (canceled)

78. A method for determining the TNM stage of a prostate cancer in a subject, the method comprising
i) administering to the subject a dose of between about 100 MBq to about 300 MBq of a compound of Formula (I) or a salt thereof complexed with ⁶⁴Cu; 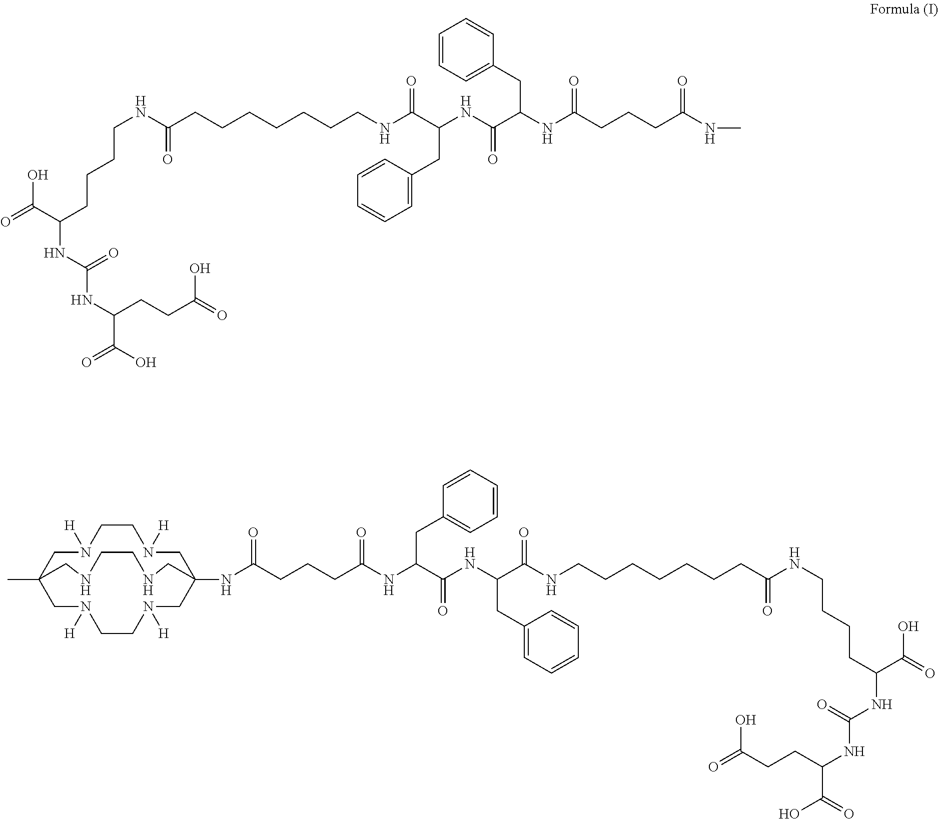
ii) imaging of the subject by PET imaging; and
iii) determining the TNM stage of the cancer on the basis of one or more images obtained in step ii) and the ISUP grade of the tumour;
wherein the ISUP grade of the tumour is determined prior to i) to iii).

79. The method according to claim 78, wherein the imaging of the subject by PET imaging is at a time of about 8 hours after administration of the compound of Formula (I).

80. The method according to claim 78, wherein the imaging of the subject by PET imaging is at a time of about 10 hours, about 12 hours, about 16 hours, about 20 hours, about 24 hours, about 28 hours, about 32 hours or about 36 hours after administration of the compound of Formula (I).

81. (canceled)

82. The method according to claim 78, wherein the imaging of the subject by PET imaging identifies one or more lesions associated with the prostate cancer.

83. The method according to claim 78, wherein the method further comprises determining a tumor-to-background ratio (TTBR) of a lesion visualised by imaging, wherein the tumor-to-background ratio (TTBR) of a lesion associated with the cancer in a the subject as detected by imaging in ii) is more than about 40.

84.-85. (canceled)

86. The method according to claim 78, wherein the ISUP grade of the tumour is Grade 1, Grade 2, Grade 3, Grade 4 or Grade 5.

87. The method according to claim 78, wherein the ISUP grade of the tumour is determined by calculation of Gleason Score.

88. The method according to claim 87, wherein the Gleason Score is 6, 7, 8, 9 or 10.

89. The method according to claim 78, wherein the TNM stage of the prostate cancer is selected from one or more of TX, T0, T1, T2, T3, T4, NX, N0, N1, N2, N3, MX, M0 or M1.

90.-119. (canceled)
